SEQUENCE LISTING (106 sequences)

SEQ ID NO: 1 - protein, 121 aa, synthetic construct
EVQLLESGGGLVQPGGSLRLSCAASGFTFGSYYMGWVRQAPGKGLEWVSYISGYSGYTYYADSVKGRFTISRDNSKNTLYLQMNSLRAEDTAVYYCARFSSPAGYPRMDYWGQGTLVTVSS

SEQ ID NO: 2 - protein, 106 aa, synthetic construct
DIQMTQSPSSLSASVGDHVTITCRASQSISSYLNWYQQKPGKAPKLLIYAASSLQSGVPSRFSGSGSGTDFTLTISSLQPEDFATYYCQQYYTLPTFGQGTKLEIK

SEQ ID NO: 3 - protein, 119 aa, synthetic construct
EVQLLESGGGLVQPGGSLRLSCAASGFTFGSYGMSWVRQAPGKGLEWVSYISYSGSYTYYADSVKGRFTISRDNSKNTLYLQMNSLRAEDTAVYYCARGADYYSVLDYWGQGTLVTVSS

SEQ ID NO: 4 - protein, 108 aa, synthetic construct
DIQMTQSPSSLSASVGDHVTITCRASQSISSYLNWYQQKPGKAPKLLIYAASSLQSGVPSRFSGSGSGTDFTLTISSLQPEDFATYYCQQFSTSPPSTFGQGTKLEIK

SEQ ID NO: 5 - protein, 120 aa, synthetic construct
EVQLLESGGGLVQPGGSLRLSCAASGFTFGGYGMSWVRQAPGKGLEWVSYIGYGGSYTYYADSVKGRFTISRDNSKNTLYLQMNSLRAEDTAVYYCARDGAGSYGFFDYWGQGTLVTVSS

SEQ ID NO: 6 - protein, 108 aa, synthetic construct
DIQMTQSPSSLSASVGDHVTITCRASQSISSYLNWYQQKPGKAPKLLIYAASSLQSGVPSRFSGSGSGTDFTLTISSLQPEDFATYYCQQFYTNPLLTFGQGTKLEIK

SEQ ID NO: 7 - protein, 118 aa, synthetic construct
EVQLLESGGGLVQPGGSLRLSCAASGFTFSYSYMYWVRQAPGKGLEWVSSISSSGGSTSYADSVKGRFTISRDNSKNTLYLQMNSLRAEDTAVYYCARPNVDSYMDYWGQGTLVTVSS

SEQ ID NO: 8 - protein, 108 aa, synthetic construct
DIQMTQSPSSLSASVGDHVTITCRASQSISSYLNWYQQKPGKAPKLLIYAASSLQSGVPSRFSGSGSGTDFTLTISSLQPEDFATYYCQQSYIYLPFTFGQGTKLEIK

SEQ ID NO: 9 - protein, 117 aa, synthetic construct
QVQLQQSGAELVRPGTSVKMSCKAAGYTFTNYWIGWVKQRPGHGLEWIGDIYPGGGYTHHNEKFKGKATLTADTSSSTAYMQLSSLTSEDSAIYYCARSVLPGFAYWGQGTLVTVSA

SEQ ID NO: 10 - protein, 107 aa, synthetic construct
DIVMTQSHKFMSTSVGDRVSITCKASQDVNTAVAWCQQKPGQSPKLLIYSASYRFTGVPDRFTGSGSGTDFTFTISSVQAEDLAVYFCQQHYSTPYTFGGGTKLEIK

SEQ ID NO: 11 - protein, 7 aa, synthetic construct
GFTFGSY

SEQ ID NO: 12 - protein, 5 aa, synthetic construct
SYYMG

SEQ ID NO: 13 - protein, 8 aa, synthetic construct
GFTFGSYY

SEQ ID NO: 14 - protein, 6 aa, synthetic construct
SGYSGY

SEQ ID NO: 15 - protein, 17 aa, synthetic construct
YISGYSGYTYYADSVKG

SEQ ID NO: 16 - protein, 8 aa, synthetic construct
ISGYSGYT

SEQ ID NO: 17 - protein, 12 aa, synthetic construct
FSSPAGYPRMDY

SEQ ID NO: 18 - protein, 12 aa, synthetic construct
FSSPAGYPRMDY

SEQ ID NO: 19 - protein, 14 aa, synthetic construct
ARFSSPAGYPRMDY

SEQ ID NO: 20 - protein, 11 aa, synthetic construct
RASQSISSYLN

SEQ ID NO: 21 - protein, 11 aa, synthetic construct
RASQSISSYLN

SEQ ID NO: 22 - protein, 6 aa, synthetic construct
QSISSY

SEQ ID NO: 23 - protein, 7 aa, synthetic construct
AASSLQS

SEQ ID NO: 24 - protein, 7 aa, synthetic construct
AASSLQS

SEQ ID NO: 25 - skipped

SEQ ID NO: 26 - protein, 8 aa, synthetic construct
QQYYTLPT

SEQ ID NO: 27 - protein, 8 aa, synthetic construct
QQYYTLPT

SEQ ID NO: 28 - protein, 8 aa, synthetic construct
QQYYTLPT

SEQ ID NO: 29 - protein, 7 aa, synthetic construct
GFTFGSY

SEQ ID NO: 30 - protein, 5 aa, synthetic construct
SYGMS

SEQ ID NO: 31 - protein, 8 aa, synthetic construct
GFTFGSYG

SEQ ID NO: 32 - protein, 6 aa, synthetic construct
SYSGSY

SEQ ID NO: 33 - protein, 17 aa, synthetic construct
YISYSGSYTYYADSVKG

SEQ ID NO: 34 - protein, 8 aa, synthetic construct
ISYSGSYT

SEQ ID NO: 35 - protein, 10 aa, synthetic construct
GADYYSVLDY

SEQ ID NO: 36 - protein, 10 aa, synthetic construct
GADYYSVLDY

SEQ ID NO: 37 - protein, 12 aa, synthetic construct
ARGADYYSVLDY

SEQ ID NO: 38 - protein, 11 aa, synthetic construct
RASQSISSYLN

SEQ ID NO: 39 - protein, 11 aa, synthetic construct
RASQSISSYLN

SEQ ID NO: 40 - protein, 6 aa, synthetic construct
QSISSY

SEQ ID NO: 41 - protein, 7 aa, synthetic construct
AASSLQS

SEQ ID NO: 42 - protein, 7 aa, synthetic construct
AASSLQS

SEQ ID NO: 43 - skipped

SEQ ID NO: 44 - protein, 10 aa, synthetic construct
QQFSTSPPST

SEQ ID NO: 45 - protein, 10 aa, synthetic construct
QQFSTSPPST

SEQ ID NO: 46 - protein, 10 aa, synthetic construct
QQFSTSPPST

SEQ ID NO: 47 - protein, 7 aa, synthetic construct
GFTFGGY

SEQ ID NO: 48 - protein, 5 aa, synthetic construct
GYGMS

SEQ ID NO: 49 - protein, 8 aa, synthetic construct
GFTFGGYG

SEQ ID NO: 50 - protein, 6 aa, synthetic construct
GYGGSY

SEQ ID NO: 51 - protein, 17 aa, synthetic construct
YIGYGGSYTYYADSVKG

SEQ ID NO: 52 - protein, 8 aa, synthetic construct
IGYGGSYT

SEQ ID NO: 53 - protein, 11 aa, synthetic construct
DGAGSYGFFDY

SEQ ID NO: 54 - protein, 11 aa, synthetic construct
DGAGSYGFFDY

SEQ ID NO: 55 - protein, 13 aa, synthetic construct
ARDGAGSYGFFDY

SEQ ID NO: 56 - protein, 11 aa, synthetic construct
RASQSISSYLN

SEQ ID NO: 57 - protein, 11 aa, synthetic construct
RASQSISSYLN

SEQ ID NO: 58 - protein, 6 aa, synthetic construct
QSISSY

SEQ ID NO: 59 - protein, 7 aa, synthetic construct
AASSLQS

SEQ ID NO: 60 - protein, 7 aa, synthetic construct
AASSLQS

SEQ ID NO: 61 - skipped

SEQ ID NO: 62 - protein, 10 aa, synthetic construct
QQFYTNPLLT

SEQ ID NO: 63 - protein, 10 aa, synthetic construct
QQFYTNPLLT

SEQ ID NO: 64 - protein, 10 aa, synthetic construct
QQFYTNPLLT

SEQ ID NO: 65 - protein, 7 aa, synthetic construct
GFTFSYS

SEQ ID NO: 66 - protein, 5 aa, synthetic construct
YSYMY

SEQ ID NO: 67 - protein, 8 aa, synthetic construct
GFTFSYSY

SEQ ID NO: 68 - protein, 6 aa, synthetic construct
SSSGGS

SEQ ID NO: 69 - protein, 17 aa, synthetic construct
SISSSGGSTSYADSVKG

SEQ ID NO: 70 - protein, 8 aa, synthetic construct
ISSSGGST

SEQ ID NO: 71 - protein, 9 aa, synthetic construct
PNVDSYMDY

SEQ ID NO: 72 - protein, 9 aa, synthetic construct
PNVDSYMDY

SEQ ID NO: 73 - protein, 11 aa, synthetic construct
ARPNVDSYMDY

SEQ ID NO: 74 - protein, 11 aa, synthetic construct
RASQSISSYLN

SEQ ID NO: 75 - protein, 11 aa, synthetic construct
RASQSISSYLN

SEQ ID NO: 76 - protein, 6 aa, synthetic construct
QSISSY

SEQ ID NO: 77 - protein, 7 aa, synthetic construct
AASSLQS

SEQ ID NO: 78 - protein, 7 aa, synthetic construct
AASSLQS

SEQ ID NO: 79 - skipped

SEQ ID NO: 80 - protein, 10 aa, synthetic construct
QQSYIYLPFT

SEQ ID NO: 81 - protein, 10 aa, synthetic construct
QQSYIYLPFT

SEQ ID NO: 82 - protein, 10 aa, synthetic construct
QQSYIYLPFT

SEQ ID NO: 83 - protein, 7 aa, synthetic construct
GYTFTNY

SEQ ID NO: 84 - protein, 5 aa, synthetic construct
NYWIG

SEQ ID NO: 85 - protein, 8 aa, synthetic construct
GYTFTNYW

SEQ ID NO: 86 - protein, 6 aa, synthetic construct
YPGGGY

SEQ ID NO: 87 - protein, 17 aa, synthetic construct
DIYPGGGYTHHNEKFKG

SEQ ID NO: 88 - protein, 8 aa, synthetic construct
IYPGGGYT

SEQ ID NO: 89 - protein, 8 aa, synthetic construct
SVLPGFAY

SEQ ID NO: 90 - protein, 8 aa, synthetic construct
SVLPGFAY

SEQ ID NO: 91 - protein, 10 aa, synthetic construct
ARSVLPGFAY

SEQ ID NO: 92 - protein, 11 aa, synthetic construct
KASQDVNTAVA

SEQ ID NO: 93 - protein, 11 aa, synthetic construct
KASQDVNTAVA

SEQ ID NO: 94 - protein, 6 aa, synthetic construct
QDVNTA

SEQ ID NO: 95 - protein, 7 aa, synthetic construct
SASYRFT

SEQ ID NO: 96 - protein, 7 aa, synthetic construct
SASYRFT

SEQ ID NO: 97 - skipped

SEQ ID NO: 98 - protein, 9 aa, synthetic construct
QQHYSTPYT

SEQ ID NO: 99 - protein, 9 aa, synthetic construct
QQHYSTPYT

SEQ ID NO: 100 - protein, 9 aa, synthetic construct
QQHYSTPYT

SEQ ID NO: 101 - protein, 23 aa, synthetic construct
DADDNVSFRWEALGNTLSSQPNL

SEQ ID NO: 102 - protein, 16 aa, synthetic construct
SVTLPLEFPAGEKVNF

SEQ ID NO: 103 - protein, 29 aa, synthetic construct
AFIVPHETKSPEIHVTNPKQGKRLNFTQS

SEQ ID NO: 104 - protein, 24 aa, synthetic construct
KMEDTGSYRAQISTKTSAKLSSYT

SEQ ID NO: 105 - protein, 332 aa, Homo sapiens
MLWLFQSLLFVFCFGPGNVVSQSSLTPLMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMILFMVSGICIVFGFIILLLLVLRKRRDSLSLSTQRTQGPAESARNLEYVSVSPTNNTVYASVTHSNRETEIWTPRENDTITIYSTINHSKESKPTFSRATALDNVV

SEQ ID NO: 106 - protein, 211 aa, synthetic construct
QSSLTPLMVNGILGESVTLPLEFPAGEKVNFITWLFNETSLAFIVPHETKSPEIHVTNPKQGKRLNFTQSYSLQLSNLKMEDTGSYRAQISTKTSAKLSSYTLRILRQLRNIQVTNHSQLFQNMTCELHLTCSVEDADDNVSFRWEALGNTLSSQPNLTVSWDPRISSEQDYTCIAENAVSNLSFSVSAQKLCEDVKIQYTDTKMHHHHHH

(19) United States
(12) Patent Application Publication
(10) Pub. No.: US 2026/0356940 A1
(43) Pub. Date: Nov. 12, 2026

(54) ANTIBODIES AND USES THEREOF

(71) Applicant: LEAD BIOLOGICS INTERNATIONAL AB, Lund (SE)

(72) Inventors: Niklas Landberg, Lund (SE); Thoas Fioretos, Lund (SE); Carl Sandén, Lund (SE)

(21) Appl. No.: 19/485,316

(22) PCT Filed: May 23, 2024
(86) PCT No.: PCT/EP2024/064282

§ 371 Date: Nov. 18, 2025

(30) Foreign Application Priority Data
May 23, 2023 (SE) 2350624.9

(51) Int. Cl. C07K 16/28; A61P 35/00

(52) U.S. Cl. CPC C07K 16/2803; A61P 35/00

(57) ABSTRACT

Described is an antibody or an antigen-binding fragment thereof that binds specifically to Signaling Lymphocytic Activating Molecule Family Member 6 (SLAMF6), wherein the antibody or antigen-binding fragment thereof is capable of inducing immune mediated killing of tumour cells. Said agent can be useful in the treatment and/or diagnosis of a cancer.

### FIELD OF THE INVENTION

The invention relates to an antibody or an antigen-binding fragment thereof that binds specifically to Signaling Lymphocytic Activating Molecule Family Member 6 (SLAMF6), wherein the antibody or antigen-binding fragment thereof is capable of inducing immune mediated killing of tumour cells. Said agent can be useful in the treatment and/or diagnosis of a myeloid malignancy and/or neoplasm, for example Acute Myeloid Leukemia (AML).

### BACKGROUND

Acute myeloid leukemia (AML) is a disease initiated and maintained by leukemia stem cells, which produce large numbers of blast cells that disrupt the normal functions of the blood system. The current chemotherapy-based treatment regimens are efficient at eliminating the blast cells but not the leukemic stem cells, which commonly survive treatment and induce therapy-resistant relapses. This treatment is also associated with severe side effects that can be life-threatening. Consequently, AML has a very poor prognosis with a 5-year survival rate of less than 30%, making it one of the deadliest forms of cancer (Dohner et al, 2022). Hence, there is a strong need to develop more efficient therapies with less side effects.

The last decade has seen an emergence of immune-modulatory therapies that have revolutionized the treatment of many solid tumors such as melanoma and lung and urinary tract cancer (Robert, 2020). AML also displays multiple characteristics of immune evasion, including tumor-mediated dysregulation of both NK cells and multiple T cell compartments. However, the first generation of checkpoint inhibitors directed against PD-1, PD-L1, CTLA-4 and CD47 has only been mildly effective, suggesting that there may be additional mechanisms which need to be modulated for successful immunotherapy in AML (Stahl & Goldberg, 2019).

To identify novel therapeutic targets on AML stem cells, the inventors previously performed a surface marker screen on cells from patients with TP53 mutations, which is the AML subtype with the worst prognosis. Using an array of PE-conjugated antibodies against human cell surface markers (Legend Screen, BioLegend), we could determine the expression of 362 such proteins on candidate leukemia stem cells and corresponding primitive cells (CD34+CD38−) from normal bone marrow.

By this approach the inventors previously discovered that the cell surface marker SLAMF6 is aberrantly expressed on primitive AML cells. SLAMF6 is normally only expressed on lymphocytes such as B, T and NK cells (Yigit et al, Clin Immunol, 2018), and has never before been detected on human stem cells or myeloid progenitor cells, or shown to be aberrantly expressed in any cancer type. The inventors next performed an extensive characterization of SLAMF6 expression in a larger cohort of 42 AML cases by FACS, demonstrating that SLAMF6 is expressed in the majority of cases and that the expression is not restricted to the TP53-mutated subgroup but includes patients with all major genetic aberrations.

SLAMF6 has recently been identified as a novel immune checkpoint regulating T cell activation and exhaustion (Hajaj et al, eLife, 2020). It was recently shown that targeting SLAMF6 on exhausted T cells could reactivate them and thus induce killing of leukemia cells (Yigit et al., 2019, Cancer Immunology Research). It has also been shown that SLAMF6 mediates NK cell activity (Wu et al., 2016, Nature Immunology).

The inventors have previously shown that CRISPR-mediated knockout of SLAMF6 in AML cells leads to increased activation of interacting T cells and subsequent killing of the AML cells (WO 2021/111005).

The inventors postulated that modulating the SLAMF6-SLAMF6 interaction using therapeutic antibodies could unleash a surprising anti-tumour response, analogous to PD-1/PD-L1 inhibitors which have revolutionized the treatment of multiple cancer types.

To be able to induce this effect with a therapeutic agent, the inventors have developed herein several SLAMF6 specific antibodies by phage-display technology as well as by mouse immunizations. In co-culture systems in vitro, the inventors have identified several candidate antibodies with remarkable effects on immune cell (e.g. T cell) activation and tumour cell killing. The inventors have also shown for the first time that various cell lines and tissue sections from solid tumours express SLAMF6, indicating that the antibodies disclosed herein are also useful in the treatment of said solid tumours.

### SUMMARY OF THE INVENTION

A first aspect of the invention provides an antibody or an antigen-binding fragment thereof that binds specifically to Signaling Lymphocytic Activating Molecule Family Member 6 (SLAMF6), wherein the antibody or antigen-binding fragment thereof is capable of inducing immune mediated killing of tumour cells.

By “immune mediated killing of tumour cells” we include any mechanism by which the antibody or antigen-binding fragment activates the immune system and/or can kill tumour cells. In some embodiments, this includes activation of immune cells (e.g. T cells, B cells, and/or NK cells) that express SLAMF6. For example, this may be mediated by interfering with SLAMF6-SLAMF6 interactions between immune cells or between immune cells and tumour cells, that leads to immune cell activation or killing. This also includes direct killing of tumour cells that express SLAMF6, for example by antibody dependent cellular cytotoxicity (ADCC), antibody dependent cellular phagocytosis (ADCP), and activation of the complement system.

The inventors have herein shown that SLAMF6 is expressed on both immune cells and cancer cells. The antibodies disclosed herein can therefore target SLAMF6-SLAMF6 interactions between cancer cells and immune cells, and between immune cells themselves. It has also been shown for the first time that SLAMF6 is expressed on the surface of solid tumour cells (as well as non-solid tumour cells), meaning the antibodies described herein will be able to act against said solid tumour cells by both immune cell mediated and direct killing of cancer cells via antibody binding to cancer cells (e.g. via ADCC).

The advantage of the antibodies described herein compared to previous SLAMF6 antibodies is their ability to induce immune mediated killing of tumour cells by a variety of mechanisms, both immune cell mediated mechanisms (e.g. T cell activation and cell killing) and direct antibody mediated mechanisms. This means that the antibodies disclosed herein have a greater anti-cancer therapeutic potential, in terms of both the types of cancer that can be targeted and the level of efficacy achieved. In some embodiments, the antibodies disclosed herein have a superior therapeutic effect (e.g. a greater degree of tumour cell killing) compared to previously disclosed SLAMF6 therapeutic antibodies.

By “Signaling Lymphocytic Activating Molecule Family Member 6” and “SLAMF6” we include any SLAMF6 protein. We specifically include the human SLAMF6 protein, for example as described in UniProtKB/Swiss-Prot Accession No. Q96DU3. SLAMF6 is also known in the scientific literature as Activating NK Receptor; NK-T-B-Antigen; NTB-A; KALI; Natural Killer-, T- And B-Cell Antigen; NTBA Receptor; CD352 Antigen; SF2000; CD352; KALIb; Ly108; and NTBA. SLAMF6 is typically expressed on the surface of NK cells, T cells, B cells and eosinophils.

The sequence of human SLAMF6 is as follows:

(SEQ ID NO. 105)
MLWLFQSLLFVFCFGPGNVVSQSSLTPLMVNGILGESVTLPLEFPAGEKV
NFITWLFNETSLAFIVPHETKSPEIHVTNPKQGKRLNFTQSYSLQLSNLK
MEDTGSYRAQISTKTSAKLSSYTLRILRQLRNIQVTNHSQLFQNMTCELH
LTCSVEDADDNVSFRWEALGNTLSSQPNLTVSWDPRISSEQDYTCIAENA
VSNLSFSVSAQKLCEDVKIQYTDTKMILFMVSGICIVFGFIILLLLVLRK
RRDSLSLSTQRTQGPAESARNLEYVSVSPTNNTVYASVTHSNRETEIWTP
RENDTITIYSTINHSKESKPTFSRATALDNVV

In some preferred embodiments, the antibody or antigen-binding fragment thereof binds specifically to human SLAMF6.

By “binds specifically” we mean that the antibody or antigen binding fragment binds SLAMF6 more strongly than other proteins, and binds SLAMF6 more strongly than other non-SLAMF6 specific antibodies.

In some embodiments, the antibody or antigen-binding fragment thereof is capable of activating immune cells. In some embodiments, the antibody or antigen-binding fragment thereof is capable of binding to and activating immune cells. In some embodiments, the antibody or antigen-binding fragment is capable of binding to SLAMF6 on the surface of immune cells, and activates said immune cells. This may be achieved by binding directly to SLAMF6 on the surface of the immune cell and preventing its interaction with SLAMF6 on the surface of another immune cell and/or a tumour cell. In some other embodiments, the antibody or antigen-binding fragment is capable of binding to SLAMF6 on the surface of tumour cells, and activates immune cells by preventing their interaction with SLAMF6 on the surface of said tumour cell.

In some embodiments, the antibody or antigen-binding fragment thereof is capable of activating T cells. In some embodiments, the antibody or antigen-binding fragment thereof is capable of binding to and activating T cells. In some embodiments, the antibody or antigen-binding fragment is capable of binding to SLAMF6 on the surface of T cells, and activates said T cells. This may be achieved by binding directly to SLAMF6 on the surface of the T cell and preventing its interaction with SLAMF6 on the surface of another immune cell and/or a tumour cell. In some other embodiments, the antibody or antigen-binding fragment is capable of binding to SLAMF6 on the surface of tumour cells, and activates T cells by preventing their interaction with SLAMF6 on the surface of said tumour cell.

In some embodiments, the antibody or antigen-binding fragment thereof is capable of activating B cells. In some embodiments, the antibody or antigen-binding fragment thereof is capable of binding to and activating B cells. In some embodiments, the antibody or antigen-binding fragment is capable of binding to SLAMF6 on the surface of B cells, and activates said B cells. This may be achieved by binding directly to SLAMF6 on the surface of the B cell and preventing its interaction with SLAMF6 on the surface of another immune cell and/or a tumour cell. In some other embodiments, the antibody or antigen-binding fragment is capable of binding to SLAMF6 on the surface of tumour cells, and activates B cells by preventing their interaction with SLAMF6 on the surface of said tumour cell.

In some embodiments, the antibody or antigen-binding fragment thereof is capable of activating NK cells. In some embodiments, the antibody or antigen-binding fragment thereof is capable of binding to and activating NK cells. In some embodiments, the antibody or antigen-binding fragment is capable of binding to SLAMF6 on the surface of NK cells, and activates said NK cells. This may be achieved by binding directly to SLAMF6 on the surface of the NK cell and preventing its interaction with SLAMF6 on the surface of another immune cell and/or a tumour cell. In some other embodiments, the antibody or antigen-binding fragment is capable of binding to SLAMF6 on the surface of tumour cells, and activates NK cells by preventing their interaction with SLAMF6 on the surface of said tumour cell.

By “capable of activating T/B/NK cells” we include that the antibody or antigen-binding fragment thereof is capable of enhancing the number, activity and/or killing of T/B/NK cells compared to a state where the antibody or antigen-binding fragment is absent, or where a control antibody or antigen binding fragment (i.e. that does not bind specifically to SLAMF6) is present. In some embodiments, the antibody or antigen binding fragment thereof activates T/B/NK cells.

For example, the antibody or antigen-binding fragment thereof may activate T cells by preventing T cell inhibition, i.e. by preventing the initiation of an inhibitory T cell pathway. In some other examples, the antibody or antigen-binding fragment thereof may activate T cells by actively activating T cells, i.e. by initiating an activatory T cell pathway. In some other embodiments, the antibody or antigen-binding fragment may activate T cells by inhibiting or reversing T cell exhaustion.

“Enhancing the number and/or activity of T cells” may include increasing the number of T cells present and/or increasing the activity of the T cells that are present compared to a control. In some embodiments, the control is when no antibody is present or when an antibody that does not specifically bind SLAMF6 is present (e.g. an isotype control). In some embodiments, by “number of T cells present” we mean the number of activated T cells present.

The increased activity of the T cells present may include the increased cytotoxic activity of the T cells and/or the increased ability of the T cells to activate other effector cells (e.g. by activating increased numbers of cytotoxic T cells, B cells, NK cells and/or macrophages). In some embodiments, the increased cytotoxic activity of T cells includes increased expression of IFN-gamma, TNF-alpha, granzyme B, and/or IL-2.

“Enhancing the number and/or activity of B cells” may include increasing the number of B cells present and/or increasing the activity of the B cells that are present compared to a control. In some embodiments, the control is when no antibody is present or when an antibody that does not specifically bind SLAMF6 is present (e.g. an isotype control). In some embodiments, by “number of B cells present” we mean the number of activated B cells present.

The increased activity of the B cells present may include the increased ability of said B cells to produce antibodies specific to tumour cell antigens, thereby increasing the ability of the B cells to mediate direct tumour cell killing.

“Enhancing the number and/or activity of NK cells” may include increasing the number of NK cells present and/or increasing the activity of the NK cells that are present compared to a control. In some embodiments, the control is when no antibody is present or when an antibody that does not specifically bind SLAMF6 is present (e.g. an isotype control). In some embodiments, by “number of NK cells present” we mean the number of activated NK cells present.

The increased activity of the NK cells present may include the increased ability of said NK cells to produce effector molecules, thereby increasing the ability of the NK cells to mediate direct tumour cell killing. In some embodiments, the effector molecules include cytokines and chemokines (e.g. IFN-γ, TNF-α, GM-CSF, IL-10, IL-5, IL-13 MIP-1α, MIP-1β, IL-8, and RANTES). Increased production of said cytokines and chemokines contributes to an inflammatory microenvironment that contributes further to immune mediated tumour cell killing.

In some embodiments, the number of T/B/NK cells present may increase by around 50% compared to a control. For example, the number of T/B/NK cells present may increase by around 55%, around 50%, around 45%, around 40%, around 35%, around 30%, around 25%, around 20%, around 15%, around 10%, or around 5%. In some embodiments, the number of T cells present may increase by around 50-60%, 40-50%, 30-40%, 20-30%, 10-20%, or 1-10%. In some embodiments, by “number of T cells present” we mean the number of activated T cells present.

In some embodiments, the number of T/B/NK cells present may increase by around 100% compared to a control. For example, the number of T/B/NK cells present may increase by around 95%, around 90%, around 85%, around 80%, around 75%, around 70%, around 65%, around 60%, or around 55%. In some embodiments, the number of T/B/NK cells present may increase by around 50-60%, 60-70%, 70-80%, 80-90%, or 90-100%. In some other embodiments, the number of T/B/NK cells present may increase by more than 100% compared to a control, for example by 110%, 120%, 130%, 140%, 150%, 160%, 170%, 180%, 190%, or 200%.

In some embodiments, the antibody or antigen binding fragment thereof is capable of increasing the number or proportion of T cells that are CD25+ and/or CD69+ compared to a control (e.g. no antibody or an isotype control).

In some embodiments, the proportion of T cells that are CD25+ and/or CD69+ increase by around 5% compared to a control. In some embodiments, the proportion of T cells that are CD25+ and/or CD69+ increase by around 4.5%, 4%, 3.5%, 3%, 2.5%, 2%, 1.5%, 1%, or 0.5% compared to a control. In some preferred embodiments, the proportion of T cells that are CD25+ and/or CD69+ increase by around 3% compared to a control.

By “T cells” we include both helper T cells and cytotoxic T cells, i.e. both CD4+ and CD8+ T cells. In some preferred embodiments, the T cells are CD4+ T cells. In other embodiments, the T cells are CD8+ T cells.

In some embodiments, the antibody or antigen binding fragment thereof induces increased T cell mediated cell killing. In some embodiments, this is in comparison to a state where the antibody or antigen-binding fragment is absent, or where a control antibody or antigen binding fragment (i.e. that does not bind specifically to SLAMF6) is present.

By “increased T cell mediated cell killing” we mean that the antibody or antigen binding fragment thereof induces cell death via a T cell mediated mechanism, and that this is increased compared to a state where the antibody or antigen-binding fragment is absent, or where a control antibody or antigen binding fragment (i.e. that does not bind specifically to SLAMF6) is present.

For example, the antibody or antigen binding fragment thereof may recruit T cells to target cells (e.g. the pathological stem cells and/or progenitor cells), activate T cells, or induce T cell-mediated apoptosis in the target cells via mechanisms known in the art (e.g. release of cytolytic granules, release of cytokines that recruit other effector cells, etc). Alternatively, or additionally, the antibody or antigen binding fragment thereof may prevent a SLAMF6-mediated response that would otherwise prevent T cell function and/or activation, for example by masking or blocking the interaction between SLAMF6 expressing cells and T cells. Similarly, the SLAMF6 binding agent may recruit, activate or otherwise stimulate immune cells, such as NK cells, for increased cell killing effects.

In some embodiments, the antibody or antigen-binding fragment thereof is capable of directly binding to and killing tumour cells.

In some embodiments, the antibodies disclosed herein are capable of inducing the following upon binding to tumour cells expressing SLAMF6:
a) antibody-dependent cellular cytotoxicity (ADCC);
b) antibody-dependent cellular phagocytosis (ADCP); and/or
c) complement-dependent cytotoxicity (CDC).

By ADCC we mean wherein the antibodies bind to a target tumour cell expressing SLAMF6 and, via recognition by immune cells (e.g. NK cells, macrophages, neutrophils, and eosinophils), trigger apoptosis of the SLAMF6 expressing tumour cell. By ADCP we mean wherein the antibodies bind to a target tumour cell expressing SLAMF6 and, via recognition by macrophages or monocytes, trigger phagocytosis of the SLAMF6 expressing tumour cell. By CDC we mean the antibodies bind to a target cell expressing SLAMF6 and, via recognition by C1q, trigger the complement cascade which leads to formation of the membrane attack complex and lysis of the SLAMF6 expressing tumour cell.

Therefore, it has been shown herein that the antibodies of the present invention are capable of modulating the immune system in multiple different ways-both by activating immune effector cells and by enhancing direct killing of tumour cells via the mechanisms described above.

By “biological activity of SLAMF6” we include any interaction or signalling event which involves SLAMF6 on pathological stem cells and/or progenitor cells as well as more mature neoplastic cells (i.e. tumour cells).

Such inhibition of the biological activity of SLAMF6 by an antibody or antigen binding fragment of the invention may be in whole or in part. For example, the agent may inhibit the biological activity of SLAMF6 by at least 10%, preferably at least 20%, 30%, 40%, 50%, 60%, 70%, 80% or 90%, and most preferably by 100% compared to the biological activity of SLAMF6 in pathological stem cells and/or progenitor cells which have not been exposed to the agent. In a preferred embodiment, the agent is capable of inhibiting the biological activity of SLAMF6 by 50% or more compared to the biological activity of SLAMF6 in pathological stem cells and/or progenitor cells which have not been exposed to the agent. The biological activity of SLAMF6 that is inhibited could be, for example, its self-ligand activity (SLAMF6 interacting with other SLAMF6) and/or downstream signalling. Examples of downstream signalling include, but are not limited to, recruitment and/or phosphorylation of mediators such as SAP, Fyn, EAT-2 and SHP-1.2 (reviewed in Yigit et al., 2018, Clinical Immunology). The biological activity of SLAMF6 that is inhibited could also be expression of particular gene(s).

SLAMF6 is a self-ligand and thus binds to other SLAMF6 molecules, which may be expressed on other immune cells such as NK cells, T cells, and B cells, or other target cells (e.g. cancer/tumour cells).

The antibody or antigen-binding fragment therefore in some embodiments blocks the self-ligand interaction of SLAMF6 on different cells or on the same cell.

Blocking the interaction between SLAMF6 expressed on an immune cell and a target (e.g. tumour) cell, can lead to activation of said immune cell. In some preferred embodiments, this activation may be due to the lack of SLAMF6-SLAMF6 interaction preventing the initiation of an inhibitory T cell pathway, thereby leading to increased T cell activation. In some other embodiments, the T cell activation may be due to the lack of SLAMF6-SLAMF6 interaction initiating an activatory T cell pathway, thereby leading to increased T cell activation. A similar mechanism also applies to the activation of other types of immune cell.

In some other embodiments, immune cell activation can be mediated by preventing a SLAMF6-SLAMF6 interaction between different immune cells (e.g. T, B, and/or NK cells) or between SLAMF6 molecules on the same cell.

For example, in relation to T cells, the antibody or antigen-binding fragment thereof preferably binds SLAMF6 expressed on the surface of a T cell, thereby activating the T cell.

In some other embodiments, the antibody or antigen-binding fragment thereof activates T cells by binding to SLAMF6 on the surface of an immune cell other than a T cell. For example, the antibody or antigen binding fragment thereof may bind SLAMF6 expressed on the surface of an NK cell or a B cell.

In some other embodiments, the antibody or antigen binding fragment thereof may bind SLAMF6 on the surface of a tumour cell.

By “tumour cell” we include any type of cancer cell or cancer associated cell, whether associated with a solid tumour or a liquid tumour (i.e. a blood derived cancer).

In some embodiments, the tumour cell is from a solid tumour. By “solid tumour” we mean any abnormal mass of tissue that is predominantly non-liquid. There are many types of solid tumours, and we include but do not limit to the following: sarcoma, lymphoma, blastoma, melanoma, carcinoma, lung cancer, pancreatic cancer, breast cancer, gastrointestinal cancer, head and neck cancer, brain cancer, liver cancer, ovarian cancer, prostate cancer, and cervical cancer.

In some preferred embodiments, the solid tumour is selected from the group consisting of: bowel cancer, colorectal cancer, esophageal cancer, stomach cancer, biliary tract cancer, kidney cancer, lung cancer, ovarian cancer, fallopian tube cancer, pancreatic cancer, or cancer of the soft tissues (e.g. sarcoma).

In some preferred embodiments, the solid tumour is colorectal cancer. In some preferred embodiments, the solid tumour is pancreatic cancer.

In some other embodiments, the tumour cell is from a liquid tumour. By “liquid tumour” we mean a cancer of the blood or another bodily fluid. Examples of liquid tumours are leukemias and leukemia-like diseases. In some embodiments, the tumour cell is from a myeloid malignancy and/or neoplasm or a lymphoid malignancy and/or neoplasm.

In some embodiments, the lymphoid malignancy and/or neoplasm is a myeloma (also known as multiple myeloma and plasma cell myeloma) or a lymphoma.

By “myeloid malignancy and/or neoplasm” we mean any clonal disorder of haematopoietic stem or progenitor cells as defined by the WHO classification of Haematolymphoid Tumours: Myeloid and Histiocytic/Dendritic Neoplasms (The 5th edition of the World Health Organization Classification of Haematolymphoid Tumours: Myeloid and Histiocytic/Dendritic Neoplasms. Leukemia. 2022 July; 36 (7): 1703-1719). We specifically include bone marrow and haematologic cancers such as leukemias (e.g. acute myeloid leukemia (AML), as well as leukemia-like diseases such as myeloproliferative disorders (MPD) (also referred to as myeloproliferative neoplasms (MPN), myelodysplastic syndromes and/or myelodysplastic neoplasms (MDS), myelodysplastic/myeloproliferative neoplasms (MDS/MPN), and mastocytosis.

More specifically, the myeloid malignancy and/or neoplasm may be selected from myelodysplastic syndrome and/or myelodysplastic neoplasms (MDS), myeloproliferative neoplasms (MPN), mastocytosis, and myelodysplastic/myeloproliferative neoplasms (MDS/MPN). In one particularly preferred embodiment, the myeloid malignancy and/or neoplasm is Acute Myeloid Leukemia (AML).

In some embodiments, Acute Myeloid Leukemia (AML) may also be referred to as Acute Myelogenous Leukemia.

In a further embodiment, the cells associated with a myeloid malignancy and/or neoplasm may be (i) pathological stem cells, and/or (ii) progenitor cells, and/or (iii) haematopoietic cells associated with a myeloid malignancy and/or neoplasm.

Preferably, the cells are pathological stem cells, and/or progenitor cells, and/or haematopoietic cells associated with AML and may be selected from: leukemic stem cells (LSCs), and/or blast cells, and/or haematopoietic cells derived from said LSCs or blast cells, or normal non-malignant cells from the innate or adaptive immune system (e.g., monocytes or T cells).

By “pathological stem cells” associated with a myeloid malignancy and/or neoplasm, we include stem cells which are responsible for the development of a myeloid malignancy and/or neoplasm in an individual, i.e. malignant and/or neoplastic stem cells. In particular, the pathological stem cells may be leukemic stem cells (for example acute myeloid leukemic (AML) cells. Such stem cell may be distinguished from normal haematopoietic stem cells (HSC) by their expression of the cell surface protein, C3AR (see the examples below).

By “progenitor cells” associated with a myeloid malignancy and/or neoplasm, we include cells derived from pathological stem cells which are responsible for the development of a myeloid malignancy and/or neoplasm in an individual. In particular, the progenitor cells may be leukemic progenitor cells. Such progenitor cells may be distinguished from normal haematopoietic progenitor cells by their higher expression of the cell surface protein, C3AR.

By “normal non-malignant hematopoietic cells from the innate and adaptive immune system”, we include myeloid and lymphoid progenitor cells, mature lymphoid cells (NK cells, T-cells and B-cells) and mature myeloid cells (dendritic cells, erythrocytes, macrophages/monocytes, megakaryocytes and granulocytes).

The skilled person will be aware of how to determine whether an antibody or antigen binding fragment has activated a T cell or population of T cells. For example, in vitro, this may involve a T cell activity assay. A T cell activity assay may involve one or more of: assessing proliferation; measuring up-regulation of activation markers e.g. IL2RA and/or CD25 and/or CD69; and measuring the production of effector cytokines e.g. INF-gamma and/or TNF-alpha. T cell activation may also be determined in vivo, for example by measuring the percentage of active T cells (e.g. those with a CD25+ CD69+ marker profile) from subjects treated with the antibody or antigen binding fragment described herein compared to a control antibody. T cell activation can also be observed indirectly, for example, by measuring the amount of cells targeted by the activated T cells (for example, in an AML model, this would involve measuring the number of AML cells compared to a control). In some other embodiments, T cell activation can be determined by observing T cell size and/or granularity.

The structure of SLAMF6 consists of an amino terminal IgV domain, a proximal IgC2 domain, a transmembrane domain, and the intracellular signal transduction domain.

In some embodiments, the antibody or antigen-binding fragment thereof binds specifically to the IgV domain of SLAMF6. In some embodiments, the antibody or antigen-binding fragment does not bind specifically to the IgC2 domain of SLAMF6. In some embodiments, the antibody or antigen-binding fragment thereof binds specifically to the IgV domain of SLAMF6 and exhibits T cell mediated killing of cancer cells.

In some embodiments, the antibody or antigen-binding fragment thereof binds specifically to an epitope of SLAMF6 comprising the following sequence SVTLPLEFPAGEKVNF (SEQ ID NO. 102). In some embodiments, the antibody or antigen-binding fragment thereof binds specifically to an epitope of SLAMF6 consisting of the following sequence SVTLPLEFPAGEKVNF.

In some embodiments, the antibody or antigen-binding fragment thereof binds specifically to an epitope of SLAMF6 comprising the following sequence AFIVPHETKSPEIHVTNPKQGKRLNFTQS (SEQ ID NO. 103). In some embodiments, the antibody or antigen-binding fragment thereof binds specifically to an epitope of SLAMF6 consisting of the following sequence AFIVPHETKSPEIHVTNPKQGKRLNFTQS.

In some embodiments, the antibody or antigen-binding fragment thereof binds specifically to an epitope of SLAMF6 comprising the following sequence KMEDTGSYRAQISTKTSAKLSSYT (SEQ ID NO. 104). In some embodiments, the antibody or antigen-binding fragment thereof binds specifically to an epitope of SLAMF6 consisting of the following sequence KMEDTGSYRAQISTKTSAKLSSYT.

The skilled person will be aware of how to produce antibodies directed to these epitopes, e.g. by hybridoma production.

### LB-031

In some embodiments, the antibody or antigen-binding fragment thereof comprises a heavy chain variable region comprising one or more of the CDRs of SEQ ID NO. 1.

In some embodiments, the antibody or antigen-binding fragment thereof comprises a heavy chain variable region comprising the following CDRs, when the CDRs are defined by Chothia:
a) SEQ ID NO. 11 or an amino acid sequence having at least 60% sequence identity therewith, for example at least 70%, 80%, or 90% sequence identity; and/or
b) SEQ ID NO. 14 or an amino acid sequence having at least 60% sequence identity therewith, for example at least 70%, 80%, or 90% sequence identity; and/or
c) SEQ ID NO. 17 or an amino acid sequence having at least 60% sequence identity therewith, for example at least 70%, 80%, or 90% sequence identity.

In some embodiments, the antibody or antigen-binding fragment thereof comprises a heavy chain variable region comprising the following CDRs, when the CDRs are defined by Kabat:
a) SEQ ID NO. 12 or an amino acid sequence having at least 60% sequence identity therewith, for example at least 70%, 80%, or 90% sequence identity; and/or
b) SEQ ID NO. 15 or an amino acid sequence having at least 60% sequence identity therewith, for example at least 70%, 80%, or 90% sequence identity; and/or
c) SEQ ID NO. 18 or an amino acid sequence having at least 60% sequence identity therewith, for example at least 70%, 80%, or 90% sequence identity.

In some embodiments, the antibody or antigen-binding fragment thereof comprises a heavy chain variable region comprising the following CDRs, when the CDRs are defined by IMGT:
a) SEQ ID NO. 13 or an amino acid sequence having at least 60% sequence identity therewith, for example at least 70%, 80%, or 90% sequence identity; and/or
b) SEQ ID NO. 16 or an amino acid sequence having at least 60% sequence identity therewith, for example at least 70%, 80%, or 90% sequence identity; and/or
c) SEQ ID NO. 19 or an amino acid sequence having at least 60% sequence identity therewith, for example at least 70%, 80%, or 90% sequence identity.

In some embodiments, the antibody or antigen-binding fragment thereof comprises a heavy chain variable region comprising the CDRs of SEQ ID NOs 11, 14, and 17.

In some embodiments, the antibody or antigen-binding fragment thereof comprises a heavy chain variable region comprising the CDRs of SEQ ID NOs 12, 15, and 18.

In some embodiments, the antibody or antigen-binding fragment thereof comprises a heavy chain variable region comprising the CDRs of SEQ ID NOs 13, 16, and 19.

In some embodiments, the antibody or antigen-binding fragment thereof comprises a heavy chain variable region comprising the CDRs of:
CDR-H1 of SEQ ID NOs 11, 12, or 13;
CDR-H2 of SEQ ID NOs 14, 15, or 16; and
CDR-H3 of SEQ ID NOs 17, 18, or 19.

In some embodiments, the antibody or antigen-binding fragment thereof comprises a heavy chain variable region having the amino acid sequence of SEQ ID NO: 1, or an amino acid sequence having at least 60% sequence identity therewith, for example at least 70%, 80%, or 90% sequence identity.

In some embodiments, the antibody or antigen-binding fragment thereof comprises a light chain variable region comprising one or more of the CDRs of SEQ ID NO: 2.

In some embodiments, the antibody or antigen-binding fragment thereof comprises a light chain variable region comprising the following CDRs, when the CDRs are determined by Chothia:
a) SEQ ID NO. 20 or an amino acid sequence having at least 60% sequence identity therewith, for example at least 70%, 80%, or 90% sequence identity; and/or
b) SEQ ID NO. 23 or an amino acid sequence having at least 60% sequence identity therewith, for example at least 70%, 80%, or 90% sequence identity; and/or
c) SEQ ID NO. 26 or an amino acid sequence having at least 60% sequence identity therewith, for example at least 70%, 80%, or 90% sequence identity.

In some embodiments, the antibody or antigen-binding fragment thereof comprises a light chain variable region comprising the following CDRs, when the CDRs are determined by Kabat:
a) SEQ ID NO. 21 or an amino acid sequence having at least 60% sequence identity therewith, for example at least 70%, 80%, or 90% sequence identity; and/or
b) SEQ ID NO. 24 or an amino acid sequence having at least 60% sequence identity therewith, for example at least 70%, 80%, or 90% sequence identity; and/or
c) SEQ ID NO. 27 or an amino acid sequence having at least 60% sequence identity therewith, for example at least 70%, 80%, or 90% sequence identity.

In some embodiments, the antibody or antigen-binding fragment thereof comprises a light chain variable region comprising the following CDRs, when the CDRs are determined by IMGT:
a) SEQ ID NO. 22 or an amino acid sequence having at least 60% sequence identity therewith, for example at least 70%, 80%, or 90% sequence identity; and/or
b) SEQ ID NO. 25 or an amino acid sequence having at least 60% sequence identity therewith, for example at least 70%, 80%, or 90% sequence identity; and/or
c) SEQ ID NO. 28 or an amino acid sequence having at least 60% sequence identity therewith, for example at least 70%, 80%, or 90% sequence identity.

In some embodiments, the antibody or antigen-binding fragment thereof comprises a light chain variable region comprising the CDRs of SEQ ID NOs 20, 23, and 26.

In some embodiments, the antibody or antigen-binding fragment thereof comprises a light chain variable region comprising the CDRs of SEQ ID NOs 21, 24, and 27.

In some embodiments, the antibody or antigen-binding fragment thereof comprises a light chain variable region comprising the CDRs of SEQ ID NOs 22, 25, and 28.

In some embodiments, the antibody or antigen-binding fragment thereof comprises a light chain variable region comprising the CDRs of:
CDR-L1 of SEQ ID NOs 20, 21, or 22;
CDR-L2 of SEQ ID NOs 23, 24, or 25; and
CDR-L3 of SEQ ID NOs 26, 27, or 28.

In some embodiments, the antibody or antigen-binding fragment thereof according to any of the preceding claims comprising a light chain variable region having the amino acid sequence of SEQ ID NO: 2, or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 11, 14, 17, 20, 23, and 26 or an amino acid sequence having at least 60% sequence identity therewith, for example at least 70%, 80%, or 90% sequence identity.

In some embodiments, the antibody or antigen-binding fragment thereof comprises the six CDRs of SEQ ID NOS 12, 15, 18, 21, 24, and 27 or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 13, 16, 19, 22, 25, and 28 or an amino acid sequence having at least 60% sequence identity therewith, for example at least 70%, 80%, or 90% sequence identity.

In some embodiments, the antibody or antigen-binding fragment thereof comprises a light chain variable region comprising the CDRs of:
CDR-H1 of SEQ ID NOs 11, 12, or 13;
CDR-H2 of SEQ ID NOs 14, 15, or 16;
CDR-H3 of SEQ ID NOs 17, 18, or 19;
CDR-L1 of SEQ ID NOs 20, 21, or 22;
CDR-L2 of SEQ ID NOs 23, 24, or 25; and
CDR-L3 of SEQ ID NOs 26, 27, or 28.

In some embodiments, the antibody or antigen-binding fragment thereof comprises a heavy chain variable region having the amino acid sequence of SEQ ID NO: 1 and a light chain variable region having the amino acid sequence of SEQ ID NO: 2, or an amino acid sequence having at least 60% sequence identity therewith, for example at least 70%, 80%, or 90% sequence identity.

In some embodiments, the antibody or antigen-binding fragment thereof comprises a heavy chain variable region having the amino acid sequence of SEQ ID NO: 1 and a light chain variable region having the amino acid sequence of SEQ ID NO: 2.

In some preferred embodiments, the antibody or antigen-binding fragment thereof is LB-031 as described herein.

### LB-032

In some embodiments, the antibody or antigen-binding fragment thereof comprises a heavy chain variable region comprising one or more of the CDRs of SEQ ID NO. 3.

In some embodiments, the antibody or antigen-binding fragment thereof comprises a heavy chain variable region comprising the following CDRs, when the CDRs are defined by Chothia:
a) SEQ ID NO. 29 or an amino acid sequence having at least 60% sequence identity therewith, for example at least 70%, 80%, or 90% sequence identity; and/or
b) SEQ ID NO. 32 or an amino acid sequence having at least 60% sequence identity therewith, for example at least 70%, 80%, or 90% sequence identity; and/or
c) SEQ ID NO. 35 or an amino acid sequence having at least 60% sequence identity therewith, for example at least 70%, 80%, or 90% sequence identity.

In some embodiments, the antibody or antigen-binding fragment thereof comprises a heavy chain variable region comprising the following CDRs, when the CDRs are defined by Kabat:
a) SEQ ID NO. 30 or an amino acid sequence having at least 60% sequence identity therewith, for example at least 70%, 80%, or 90% sequence identity; and/or
b) SEQ ID NO. 33 or an amino acid sequence having at least 60% sequence identity therewith, for example at least 70%, 80%, or 90% sequence identity; and/or
c) SEQ ID NO. 36 or an amino acid sequence having at least 60% sequence identity therewith, for example at least 70%, 80%, or 90% sequence identity.

In some embodiments, the antibody or antigen-binding fragment thereof comprises a heavy chain variable region comprising the following CDRs, when the CDRs are defined by IMGT:
a) SEQ ID NO. 31 or an amino acid sequence having at least 60% sequence identity therewith, for example at least 70%, 80%, or 90% sequence identity; and/or
b) SEQ ID NO. 34 or an amino acid sequence having at least 60% sequence identity therewith, for example at least 70%, 80%, or 90% sequence identity; and/or
c) SEQ ID NO. 37 or an amino acid sequence having at least 60% sequence identity therewith, for example at least 70%, 80%, or 90% sequence identity.

In some embodiments, the antibody or antigen-binding fragment thereof comprises a heavy chain variable region comprising the CDRs of SEQ ID NOs 29, 32, and 35.

In some embodiments, the antibody or antigen-binding fragment thereof comprises a heavy chain variable region comprising the CDRs of SEQ ID NOs 30, 33, and 36.

In some embodiments, the antibody or antigen-binding fragment thereof comprises a heavy chain variable region comprising the CDRs of SEQ ID NOs 31, 34, and 37.

In some embodiments, the antibody or antigen-binding fragment thereof comprises a heavy chain variable region comprising the CDRs of:
CDR-H1 of SEQ ID NOs 29, 30, or 31;
CDR-H2 of SEQ ID NOs 32, 33, or 34; and
CDR-H3 of SEQ ID NOs 35, 36, or 37.

In some embodiments, the antibody or antigen-binding fragment thereof comprises a heavy chain variable region having the amino acid sequence of SEQ ID NO: 3, or an amino acid sequence having at least 60% sequence identity therewith, for example at least 70%, 80%, or 90% sequence identity.

In some embodiments, the antibody or antigen-binding fragment thereof comprises a light chain variable region comprising one or more of the CDRs of SEQ ID NO: 4.

In some embodiments, the antibody or antigen-binding fragment thereof comprises a light chain variable region comprising the following CDRs, when the CDRs are determined by Chothia:
a) SEQ ID NO. 38 or an amino acid sequence having at least 60% sequence identity therewith, for example at least 70%, 80%, or 90% sequence identity; and/or
b) SEQ ID NO. 41 or an amino acid sequence having at least 60% sequence identity therewith, for example at least 70%, 80%, or 90% sequence identity; and/or
c) SEQ ID NO. 44 or an amino acid sequence having at least 60% sequence identity therewith, for example at least 70%, 80%, or 90% sequence identity.

In some embodiments, the antibody or antigen-binding fragment thereof comprises a light chain variable region comprising the following CDRs, when the CDRs are determined by Kabat:
a) SEQ ID NO. 39 or an amino acid sequence having at least 60% sequence identity therewith, for example at least 70%, 80%, or 90% sequence identity; and/or
b) SEQ ID NO. 42 or an amino acid sequence having at least 60% sequence identity therewith, for example at least 70%, 80%, or 90% sequence identity; and/or
c) SEQ ID NO. 45 or an amino acid sequence having at least 60% sequence identity therewith, for example at least 70%, 80%, or 90% sequence identity.

In some embodiments, the antibody or antigen-binding fragment thereof comprises a light chain variable region comprising the following CDRs, when the CDRs are determined by IMGT:
a) SEQ ID NO. 40 or an amino acid sequence having at least 60% sequence identity therewith, for example at least 70%, 80%, or 90% sequence identity; and/or
b) SEQ ID NO. 43 or an amino acid sequence having at least 60% sequence identity therewith, for example at least 70%, 80%, or 90% sequence identity; and/or
c) SEQ ID NO. 46 or an amino acid sequence having at least 60% sequence identity therewith, for example at least 70%, 80%, or 90% sequence identity.

In some embodiments, the antibody or antigen-binding fragment thereof comprises a light chain variable region comprising the CDRs of SEQ ID NOs 38, 41, and 44.

In some embodiments, the antibody or antigen-binding fragment thereof comprises a light chain variable region comprising the CDRs of SEQ ID NOs 39, 42, and 45.

In some embodiments, the antibody or antigen-binding fragment thereof comprises a light chain variable region comprising the CDRs of SEQ ID NOs 40, 43, and 46.

In some embodiments, the antibody or antigen-binding fragment thereof comprises a light chain variable region comprising the CDRs of:
CDR-L1 of SEQ ID NOs 38, 39, or 40;
CDR-L2 of SEQ ID NOs 41, 42, or 43; and
CDR-L3 of SEQ ID NOs 44, 45, or 46.

In some embodiments, the antibody or antigen-binding fragment thereof according to any of the preceding claims comprising a light chain variable region having the amino acid sequence of SEQ ID NO: 4, or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 29, 32, 35, 38, 41, and 44 or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 30, 33, 36, 39, 42, and 45 or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 31, 34, 37, 40, 43, and 46 or an amino acid sequence having at least 60% sequence identity therewith, for example at least 70%, 80%, or 90% sequence identity.

In some embodiments, the antibody or antigen-binding fragment thereof comprises a light chain variable region comprising the CDRs of:
CDR-H1 of SEQ ID NOs 29, 30, or 31;
CDR-H2 of SEQ ID NOs 32, 33, or 34;
CDR-H3 of SEQ ID NOs 35, 36, or 37;
CDR-L1 of SEQ ID NOs 38, 39, or 40;
CDR-L2 of SEQ ID NOs 41, 42, or 43; and
CDR-L3 of SEQ ID NOs 44, 45, or 46.

In some embodiments, the antibody or antigen-binding fragment thereof comprises a heavy chain variable region having the amino acid sequence of SEQ ID NO: 3 and a light chain variable region having the amino acid sequence of SEQ ID NO: 4, or an amino acid sequence having at least 60% sequence identity therewith, for example at least 70%, 80%, or 90% sequence identity.

In some embodiments, the antibody or antigen-binding fragment thereof comprises a heavy chain variable region having the amino acid sequence of SEQ ID NO: 3 and a light chain variable region having the amino acid sequence of SEQ ID NO: 4.

### LB-302

In some embodiments, the antibody or antigen-binding fragment thereof comprises a heavy chain variable region comprising one or more of the CDRs of SEQ ID NO. 5.

In some embodiments, the antibody or antigen-binding fragment thereof comprises a heavy chain variable region comprising the following CDRs, when the CDRs are defined by Chothia:
a) SEQ ID NO. 47 or an amino acid sequence having at least 60% sequence identity therewith, for example at least 70%, 80%, or 90% sequence identity; and/or
b) SEQ ID NO. 50 or an amino acid sequence having at least 60% sequence identity therewith, for example at least 70%, 80%, or 90% sequence identity; and/or
c) SEQ ID NO. 53 or an amino acid sequence having at least 60% sequence identity therewith, for example at least 70%, 80%, or 90% sequence identity.

In some embodiments, the antibody or antigen-binding fragment thereof comprises a heavy chain variable region comprising the following CDRs, when the CDRs are defined by Kabat:
a) SEQ ID NO. 48 or an amino acid sequence having at least 60% sequence identity therewith, for example at least 70%, 80%, or 90% sequence identity; and/or
b) SEQ ID NO. 51 or an amino acid sequence having at least 60% sequence identity therewith, for example at least 70%, 80%, or 90% sequence identity; and/or
c) SEQ ID NO. 54 or an amino acid sequence having at least 60% sequence identity therewith, for example at least 70%, 80%, or 90% sequence identity.

In some embodiments, the antibody or antigen-binding fragment thereof comprises a heavy chain variable region comprising the following CDRs, when the CDRs are defined by IMGT:
a) SEQ ID NO. 49 or an amino acid sequence having at least 60% sequence identity therewith, for example at least 70%, 80%, or 90% sequence identity; and/or
b) SEQ ID NO. 52 or an amino acid sequence having at least 60% sequence identity therewith, for example at least 70%, 80%, or 90% sequence identity; and/or
c) SEQ ID NO. 55 or an amino acid sequence having at least 60% sequence identity therewith, for example at least 70%, 80%, or 90% sequence identity.

In some embodiments, the antibody or antigen-binding fragment thereof comprises a heavy chain variable region comprising the CDRs of SEQ ID NOs 47, 50, and 53.

In some embodiments, the antibody or antigen-binding fragment thereof comprises a heavy chain variable region comprising the CDRs of SEQ ID NOs 48, 51, and 54.

In some embodiments, the antibody or antigen-binding fragment thereof comprises a heavy chain variable region comprising the CDRs of SEQ ID NOs 49, 52, and 55.

In some embodiments, the antibody or antigen-binding fragment thereof comprises a heavy chain variable region comprising the CDRs of:
CDR-H1 of SEQ ID NOs 47, 48, or 49;
CDR-H2 of SEQ ID NOs 50, 51, or 52; and
CDR-H3 of SEQ ID NOs 53, 54, or 55.

In some embodiments, the antibody or antigen-binding fragment thereof comprises a heavy chain variable region having the amino acid sequence of SEQ ID NO: 5, or an amino acid sequence having at least 60% sequence identity therewith, for example at least 70%, 80%, or 90% sequence identity.

In some embodiments, the antibody or antigen-binding fragment thereof comprises a light chain variable region comprising one or more of the CDRs of SEQ ID NO: 6.

In some embodiments, the antibody or antigen-binding fragment thereof comprises a light chain variable region comprising the following CDRs, when the CDRs are determined by Chothia:
a) SEQ ID NO. 56 or an amino acid sequence having at least 60% sequence identity therewith, for example at least 70%, 80%, or 90% sequence identity; and/or
b) SEQ ID NO. 59 or an amino acid sequence having at least 60% sequence identity therewith, for example at least 70%, 80%, or 90% sequence identity; and/or
c) SEQ ID NO. 62 or an amino acid sequence having at least 60% sequence identity therewith, for example at least 70%, 80%, or 90% sequence identity.

In some embodiments, the antibody or antigen-binding fragment thereof comprises a light chain variable region comprising the following CDRs, when the CDRs are determined by Kabat:
a) SEQ ID NO. 57 or an amino acid sequence having at least 60% sequence identity therewith, for example at least 70%, 80%, or 90% sequence identity; and/or
b) SEQ ID NO. 60 or an amino acid sequence having at least 60% sequence identity therewith, for example at least 70%, 80%, or 90% sequence identity; and/or
c) SEQ ID NO. 63 or an amino acid sequence having at least 60% sequence identity therewith, for example at least 70%, 80%, or 90% sequence identity.

In some embodiments, the antibody or antigen-binding fragment thereof comprises a light chain variable region comprising the following CDRs, when the CDRs are determined by IMGT:
a) SEQ ID NO. 58 or an amino acid sequence having at least 60% sequence identity therewith, for example at least 70%, 80%, or 90% sequence identity; and/or
b) SEQ ID NO. 61 or an amino acid sequence having at least 60% sequence identity therewith, for example at least 70%, 80%, or 90% sequence identity; and/or
c) SEQ ID NO. 64 or an amino acid sequence having at least 60% sequence identity therewith, for example at least 70%, 80%, or 90% sequence identity.

In some embodiments, the antibody or antigen-binding fragment thereof comprises a light chain variable region comprising the CDRs of SEQ ID NOs 56, 59, and 62.

In some embodiments, the antibody or antigen-binding fragment thereof comprises a light chain variable region comprising the CDRs of SEQ ID NOs 57, 60, and 63.

In some embodiments, the antibody or antigen-binding fragment thereof comprises a light chain variable region comprising the CDRs of SEQ ID NOs 58, 61, and 64.

In some embodiments, the antibody or antigen-binding fragment thereof comprises a light chain variable region comprising the CDRs of:
CDR-L1 of SEQ ID NOs 56, 57, or 58;
CDR-L2 of SEQ ID NOs 59, 60, or 61; and
CDR-L3 of SEQ ID NOs 62, 63, or 64.

In some embodiments, the antibody or antigen-binding fragment thereof according to any of the preceding claims comprising a light chain variable region having the amino acid sequence of SEQ ID NO: 6, or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 47, 50, 53, 56, 59, and 62 or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 48, 51, 54, 57, 60, and 63 or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 49, 52, 55, 58, 61, and 64 or an amino acid sequence having at least 60% sequence identity therewith, for example at least 70%, 80%, or 90% sequence identity.

In some embodiments, the antibody or antigen-binding fragment thereof comprises a light chain variable region comprising the CDRs of:
CDR-H1 of SEQ ID NOs 47, 48, or 49;
CDR-H2 of SEQ ID NOs 50, 51, or 52;
CDR-H3 of SEQ ID NOs 53, 54, or 55;
CDR-L1 of SEQ ID NOs 56, 57, or 58;
CDR-L2 of SEQ ID NOs 59, 60, or 61; and
CDR-L3 of SEQ ID NOs 62, 63, or 64.

In some embodiments, the antibody or antigen-binding fragment thereof comprises a heavy chain variable region having the amino acid sequence of SEQ ID NO: 5 and a light chain variable region having the amino acid sequence of SEQ ID NO: 6, or an amino acid sequence having at least 60% sequence identity therewith, for example at least 70%, 80%, or 90% sequence identity.

In some embodiments, the antibody or antigen-binding fragment thereof comprises a heavy chain variable region having the amino acid sequence of SEQ ID NO: 5 and a light chain variable region having the amino acid sequence of SEQ ID NO: 6.

### LB-116

In some embodiments, the antibody or antigen-binding fragment thereof comprises a heavy chain variable region comprising one or more of the CDRs of SEQ ID NO. 7.

In some embodiments, the antibody or antigen-binding fragment thereof comprises a heavy chain variable region comprising the following CDRs, when the CDRs are defined by Chothia:
a) SEQ ID NO. 65 or an amino acid sequence having at least 60% sequence identity therewith, for example at least 70%, 80%, or 90% sequence identity; and/or
b) SEQ ID NO. 68 or an amino acid sequence having at least 60% sequence identity therewith, for example at least 70%, 80%, or 90% sequence identity; and/or
c) SEQ ID NO. 71 or an amino acid sequence having at least 60% sequence identity therewith, for example at least 70%, 80%, or 90% sequence identity.

In some embodiments, the antibody or antigen-binding fragment thereof comprises a heavy chain variable region comprising the following CDRs, when the CDRs are defined by Kabat:
a) SEQ ID NO. 66 or an amino acid sequence having at least 60% sequence identity therewith, for example at least 70%, 80%, or 90% sequence identity; and/or
b) SEQ ID NO. 69 or an amino acid sequence having at least 60% sequence identity therewith, for example at least 70%, 80%, or 90% sequence identity; and/or
c) SEQ ID NO. 72 or an amino acid sequence having at least 60% sequence identity therewith, for example at least 70%, 80%, or 90% sequence identity.

In some embodiments, the antibody or antigen-binding fragment thereof comprises a heavy chain variable region comprising the following CDRs, when the CDRs are defined by IMGT:
a) SEQ ID NO. 67 or an amino acid sequence having at least 60% sequence identity therewith, for example at least 70%, 80%, or 90% sequence identity; and/or
b) SEQ ID NO. 70 or an amino acid sequence having at least 60% sequence identity therewith, for example at least 70%, 80%, or 90% sequence identity; and/or
c) SEQ ID NO. 73 or an amino acid sequence having at least 60% sequence identity therewith, for example at least 70%, 80%, or 90% sequence identity.

In some embodiments, the antibody or antigen-binding fragment thereof comprises a heavy chain variable region comprising the CDRs of SEQ ID NOs 65, 68, and 71.

In some embodiments, the antibody or antigen-binding fragment thereof comprises a heavy chain variable region comprising the CDRs of SEQ ID NOs 66, 69, and 72.

In some embodiments, the antibody or antigen-binding fragment thereof comprises a heavy chain variable region comprising the CDRs of SEQ ID NOs 67, 70, and 73.

In some embodiments, the antibody or antigen-binding fragment thereof comprises a heavy chain variable region comprising the CDRs of:
CDR-H1 of SEQ ID NOs 65, 66, or 67;
CDR-H2 of SEQ ID NOs 68, 69, or 70; and
CDR-H3 of SEQ ID NOs 71, 72, or 73.

In some embodiments, the antibody or antigen-binding fragment thereof comprises a heavy chain variable region having the amino acid sequence of SEQ ID NO: 7, or an amino acid sequence having at least 60% sequence identity therewith, for example at least 70%, 80%, or 90% sequence identity.

In some embodiments, the antibody or antigen-binding fragment thereof comprises a light chain variable region comprising one or more of the CDRs of SEQ ID NO: 8.

In some embodiments, the antibody or antigen-binding fragment thereof comprises a light chain variable region comprising the following CDRs, when the CDRs are determined by Chothia:
a) SEQ ID NO. 74 or an amino acid sequence having at least 60% sequence identity therewith, for example at least 70%, 80%, or 90% sequence identity; and/or
b) SEQ ID NO. 77 or an amino acid sequence having at least 60% sequence identity therewith, for example at least 70%, 80%, or 90% sequence identity; and/or
c) SEQ ID NO. 80 or an amino acid sequence having at least 60% sequence identity therewith, for example at least 70%, 80%, or 90% sequence identity.

In some embodiments, the antibody or antigen-binding fragment thereof comprises a light chain variable region comprising the following CDRs, when the CDRs are determined by Kabat:
a) SEQ ID NO. 75 or an amino acid sequence having at least 60% sequence identity therewith, for example at least 70%, 80%, or 90% sequence identity; and/or
b) SEQ ID NO. 78 or an amino acid sequence having at least 60% sequence identity therewith, for example at least 70%, 80%, or 90% sequence identity; and/or
c) SEQ ID NO. 81 or an amino acid sequence having at least 60% sequence identity therewith, for example at least 70%, 80%, or 90% sequence identity.

In some embodiments, the antibody or antigen-binding fragment thereof comprises a light chain variable region comprising the following CDRs, when the CDRs are determined by IMGT:
a) SEQ ID NO. 76 or an amino acid sequence having at least 60% sequence identity therewith, for example at least 70%, 80%, or 90% sequence identity; and/or
b) SEQ ID NO. 78 or an amino acid sequence having at least 60% sequence identity therewith, for example at least 70%, 80%, or 90% sequence identity; and/or
c) SEQ ID NO. 82 or an amino acid sequence having at least 60% sequence identity therewith, for example at least 70%, 80%, or 90% sequence identity.

In some embodiments, the antibody or antigen-binding fragment thereof comprises a light chain variable region comprising the CDRs of SEQ ID NOs 74, 77, and 80.

In some embodiments, the antibody or antigen-binding fragment thereof comprises a light chain variable region comprising the CDRs of SEQ ID NOs 75, 78, and 81.

In some embodiments, the antibody or antigen-binding fragment thereof comprises a light chain variable region comprising the CDRs of SEQ ID NOs 76, 78, and 82.

In some embodiments, the antibody or antigen-binding fragment thereof comprises a light chain variable region comprising the CDRs of:
CDR-L1 of SEQ ID NOs 74, 75, or 76;
CDR-L2 of SEQ ID NOs 77, 78, or 79; and
CDR-L3 of SEQ ID NOs 80, 81, or 82.

In some embodiments, the antibody or antigen-binding fragment thereof according to any of the preceding claims comprising a light chain variable region having the amino acid sequence of SEQ ID NO: 8, or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 65, 68, 71, 74, 77, and 80 or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 66, 69, 72, 75, 78, and 81 or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 67, 70, 73, 76, 79, and 82 or an amino acid sequence having at least 60% sequence identity therewith, for example at least 70%, 80%, or 90% sequence identity.

In some embodiments, the antibody or antigen-binding fragment thereof comprises a light chain variable region comprising the CDRs of:
CDR-H1 of SEQ ID NOs 65, 66, or 67;
CDR-H2 of SEQ ID NOs 68, 69, or 70;
CDR-H3 of SEQ ID NOs 71, 72, or 73;
CDR-L1 of SEQ ID NOs 74, 75, or 76;
CDR-L2 of SEQ ID NOs 77, 78, or 79; and
CDR-L3 of SEQ ID NOs 80, 81, or 82.

In some embodiments, the antibody or antigen-binding fragment thereof comprises a heavy chain variable region having the amino acid sequence of SEQ ID NO: 7 and a light chain variable region having the amino acid sequence of SEQ ID NO: 8, or an amino acid sequence having at least 60% sequence identity therewith, for example at least 70%, 80%, or 90% sequence identity.

In some embodiments, the antibody or antigen-binding fragment thereof comprises a heavy chain variable region having the amino acid sequence of SEQ ID NO: 7 and a light chain variable region having the amino acid sequence of SEQ ID NO: 8.

In some preferred embodiments, the antibody or antigen-binding fragment thereof is LB-116 as described herein.

### LB-20H10

In some embodiments, the antibody or antigen-binding fragment thereof comprises a heavy chain variable region comprising one or more of the CDRs of SEQ ID NO. 9.

In some embodiments, the antibody or antigen-binding fragment thereof comprises a heavy chain variable region comprising the following CDRs, when the CDRs are defined by Chothia:
a) SEQ ID NO. 83 or an amino acid sequence having at least 60% sequence identity therewith, for example at least 70%, 80%, or 90% sequence identity; and/or
b) SEQ ID NO. 86 or an amino acid sequence having at least 60% sequence identity therewith, for example at least 70%, 80%, or 90% sequence identity; and/or
c) SEQ ID NO. 89 or an amino acid sequence having at least 60% sequence identity therewith, for example at least 70%, 80%, or 90% sequence identity.

In some embodiments, the antibody or antigen-binding fragment thereof comprises a heavy chain variable region comprising the following CDRs, when the CDRs are defined by Kabat:
a) SEQ ID NO. 84 or an amino acid sequence having at least 60% sequence identity therewith, for example at least 70%, 80%, or 90% sequence identity; and/or
b) SEQ ID NO. 87 or an amino acid sequence having at least 60% sequence identity therewith, for example at least 70%, 80%, or 90% sequence identity; and/or
c) SEQ ID NO. 90 or an amino acid sequence having at least 60% sequence identity therewith, for example at least 70%, 80%, or 90% sequence identity.

In some embodiments, the antibody or antigen-binding fragment thereof comprises a heavy chain variable region comprising the following CDRs, when the CDRs are defined by IMGT:
a) SEQ ID NO. 85 or an amino acid sequence having at least 60% sequence identity therewith, for example at least 70%, 80%, or 90% sequence identity; and/or
b) SEQ ID NO. 88 or an amino acid sequence having at least 60% sequence identity therewith, for example at least 70%, 80%, or 90% sequence identity; and/or
c) SEQ ID NO. 91 or an amino acid sequence having at least 60% sequence identity therewith, for example at least 70%, 80%, or 90% sequence identity.

In some embodiments, the antibody or antigen-binding fragment thereof comprises a heavy chain variable region comprising the CDRs of SEQ ID NOs 83, 86, and 89.

In some embodiments, the antibody or antigen-binding fragment thereof comprises a heavy chain variable region comprising the CDRs of SEQ ID NOs 84, 87, and 90.

In some embodiments, the antibody or antigen-binding fragment thereof comprises a heavy chain variable region comprising the CDRs of SEQ ID NOs 85, 88, and 91.

In some embodiments, the antibody or antigen-binding fragment thereof comprises a heavy chain variable region comprising the CDRs of:
CDR-H1 of SEQ ID NOs 83, 84, or 85;
CDR-H2 of SEQ ID NOs 86, 87, or 88; and
CDR-H3 of SEQ ID NOs 89, 90, or 91.

In some embodiments, the antibody or antigen-binding fragment thereof comprises a heavy chain variable region having the amino acid sequence of SEQ ID NO: 9, or an amino acid sequence having at least 60% sequence identity therewith, for example at least 70%, 80%, or 90% sequence identity.

In some embodiments, the antibody or antigen-binding fragment thereof comprises a light chain variable region comprising one or more of the CDRs of SEQ ID NO: 10.

In some embodiments, the antibody or antigen-binding fragment thereof comprises a light chain variable region comprising the following CDRs, when the CDRs are determined by Chothia:
a) SEQ ID NO. 92 or an amino acid sequence having at least 60% sequence identity therewith, for example at least 70%, 80%, or 90% sequence identity; and/or
b) SEQ ID NO. 95 or an amino acid sequence having at least 60% sequence identity therewith, for example at least 70%, 80%, or 90% sequence identity; and/or
c) SEQ ID NO. 98 or an amino acid sequence having at least 60% sequence identity therewith, for example at least 70%, 80%, or 90% sequence identity.

In some embodiments, the antibody or antigen-binding fragment thereof comprises a light chain variable region comprising the following CDRs, when the CDRs are determined by Kabat:
a) SEQ ID NO. 93 or an amino acid sequence having at least 60% sequence identity therewith, for example at least 70%, 80%, or 90% sequence identity; and/or
b) SEQ ID NO. 96 or an amino acid sequence having at least 60% sequence identity therewith, for example at least 70%, 80%, or 90% sequence identity; and/or
c) SEQ ID NO. 99 or an amino acid sequence having at least 60% sequence identity therewith, for example at least 70%, 80%, or 90% sequence identity.

In some embodiments, the antibody or antigen-binding fragment thereof comprises a light chain variable region comprising the following CDRs, when the CDRs are determined by IMGT:
a) SEQ ID NO. 94 or an amino acid sequence having at least 60% sequence identity therewith, for example at least 70%, 80%, or 90% sequence identity; and/or
b) SEQ ID NO. 97 or an amino acid sequence having at least 60% sequence identity therewith, for example at least 70%, 80%, or 90% sequence identity; and/or
c) SEQ ID NO. 100 or an amino acid sequence having at least 60% sequence identity therewith, for example at least 70%, 80%, or 90% sequence identity.

In some embodiments, the antibody or antigen-binding fragment thereof comprises a light chain variable region comprising the CDRs of SEQ ID NOs 92, 95, and 98.

In some embodiments, the antibody or antigen-binding fragment thereof comprises a light chain variable region comprising the CDRs of SEQ ID NOs 93, 96, and 99.

In some embodiments, the antibody or antigen-binding fragment thereof comprises a light chain variable region comprising the CDRs of SEQ ID NOs 94, 97, and 100.

In some embodiments, the antibody or antigen-binding fragment thereof comprises a light chain variable region comprising the CDRs of:
CDR-L1 of SEQ ID NOs 92, 93, or 94;
CDR-L2 of SEQ ID NOs 95, 96, or 97; and
CDR-L3 of SEQ ID NOs 98, 99, or 100.

In some embodiments, the antibody or antigen-binding fragment thereof according to any of the preceding claims comprising a light chain variable region having the amino acid sequence of SEQ ID NO: 10, or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 83, 86, 89, 92, 95, and 98 or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 84, 87, 90, 93, 96, and 99 or an amino acid sequence having at least 60% sequence identity therewith, for example at least 70%, 80%, or 90% sequence identity.

In some embodiments, the antibody or antigen-binding fragment thereof comprises the six CDRs of SEQ ID NOs 85, 88, 91, 94, 97, and 100 or an amino acid sequence having at least 60% sequence identity therewith, for example at least 70%, 80%, or 90% sequence identity.

In some embodiments, the antibody or antigen-binding fragment thereof comprises a light chain variable region comprising the CDRs of:
CDR-H1 of SEQ ID NOs 83, 84, or 85;
CDR-H2 of SEQ ID NOs 86, 87, or 88;
CDR-H3 of SEQ ID NOs 89, 90, or 91;
CDR-L1 of SEQ ID NOs 92, 93, or 94;
CDR-L2 of SEQ ID NOs 95, 96, or 97; and
CDR-L3 of SEQ ID NOs 98, 99, or 100.

In some embodiments, the antibody or antigen-binding fragment thereof comprises a heavy chain variable region having the amino acid sequence of SEQ ID NO: 9 and a light chain variable region having the amino acid sequence of SEQ ID NO: 10, or an amino acid sequence having at least 60% sequence identity therewith, for example at least 70%, 80%, or 90% sequence identity.

In some embodiments, the antibody or antigen-binding fragment thereof comprises a heavy chain variable region having the amino acid sequence of SEQ ID NO: 9 and a light chain variable region having the amino acid sequence of SEQ ID NO: 10.

By “antibody” we include substantially intact antibody molecules, as well as chimeric antibodies, humanised antibodies, human antibodies (wherein at least one amino acid is mutated relative to the naturally occurring human antibodies), single chain antibodies, antibody heavy chains, antibody light chains, homodimers and heterodimers of antibody heavy and/or light chains, and antigen binding fragments and derivatives of the same.

Typical immunoglobulin molecules comprise four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

Methods and techniques for identifying CDRs within variable region amino acid sequences are well known in the art and can be used to identify CDRs within the specified antibodies or fragments amino acid sequences disclosed herein. Exemplary conventions that can be used to identify the boundaries of CDRs include, e.g., the Kabat definition, the Chothia definition, and the IMGT definition. See, for example: Kabat, “Sequences of Proteins of Immunological Interest,” National Institutes of Health, Bethesda, Md. (1991); Chothia C, Lesk a M. “Canonical structures for the hypervariable regions of immunoglobulins”. J Mol Biol. (1987) 196:901-17; Al-Lazikani et al., J. Mol. Biol. 273:927-948 (1997); and Martin et al., Proc. Natl. Acad. Sci. USA 86:9268-9272 (1989). Public databases are also available to the skilled person for identifying CDR sequences within an antibody.

CDR sequences may be defined using any one of the IMGT, Chothia, and KABAT numbering schemes, or a combination of the numbering schemes. In an embodiment, the CDRs are defined using the IMGT numbering system. In an embodiment, the CDRs are defined using the Kabat numbering system. In an embodiment, the CDRs are defined using the Chothia numbering system.

By “antigen-binding fragment” we mean a functional fragment of an antibody that is capable of binding to SLAMF6.

Preferably, the antigen-binding fragment is selected from the group consisting of Fv fragments (e.g. single chain Fv (scFv) and disulphide-bonded Fv and di-scFvs), Fab-like fragments (e.g. Fab fragments, Fab-SH, Fab′ fragments and F(ab)2 fragments), single variable domains (e.g. VH and VL domains) and domain antibodies (sdAbs, including single and dual formats [i.e. dAb-linker-dAb]).

In some embodiments, the antigen-binding fragment is selected from: scFv, Fv, Fab, F(ab′)2, Fab-SH, dsFv, VH, VL, sdAb, di-scFvs, and Fcabs.

The advantages of using antibody fragments, rather than whole antibodies, are several-fold. The smaller size of the fragments may lead to improved pharmacological properties, such as better penetration of solid tissue. Moreover, antigen-binding fragments such as Fab, Fv, ScFv and dAb antibody fragments can be expressed in and secreted from E. coli, thus allowing the production of large amounts of the said fragments.

As discussed herein, it will be appreciated by persons skilled in the art that the antibodies and antigen binding fragments of the invention may alternatively comprise variants of the above-defined sequences. The antibodies and antigen binding fragments thereof may have at least 60% sequence identity with any of the sequences disclosed herein. For example, they may have at least 60%, at least 65%, at least 70%, at least 75%, at least 80%, at least 85%, at least 90%, at least 95%, or at least 99% sequence identity.

The antibodies and antigen binding fragments thereof may alternatively be a variant of a specific sequence disclosed herein, wherein said variant comprises mutations at one or more positions relative to the parent sequence. By “mutation” we include insertions, deletions and substitutions. Accordingly a variant may be a substitution, deletion or addition variant. A variant polypeptide may comprise 1, 2, 3, 4, 5, 6, 7, 8, 9, up to 10, up to 20, up to 30, up to 40, up to 50, up to 75 or more amino acid mutations, in a preferred embodiment. The mutations may be either conservative or non-conservative. For example, conservative substitution refers to the substitution of an amino acid within the same general class (e.g. an acidic amino acid, a basic amino acid, a non-polar amino acid, a polar amino acid or an aromatic amino acid) by another amino acid within the same class. Thus, the meaning of a conservative amino acid substitution and non-conservative amino acid substitution is well known in the art.

“Deletion” variants may comprise the deletion of individual amino acids, deletion of small groups of amino acids such as 2, 3, 4 or 5 amino acids, or deletion of larger amino acid regions, such as the deletion of specific amino acid domains or other features. “Substitution” variants preferably involve the replacement of one or more amino acids with the same number of amino acids and making conservative amino acid substitutions. For example, an amino acid may be substituted with an alternative amino acid having similar properties, for example, another basic amino acid, another acidic amino acid, another neutral amino acid, another charged amino acid, another hydrophilic amino acid, another hydrophobic amino acid, another polar amino acid, another aromatic amino acid or another aliphatic amino acid. Some properties of the 20 main amino acids which can be used to select suitable substituents are as follows:

| | | | |
|---|---|---|---|
| Ala, A | aliphatic, hydrophobic, | Met, | hydrophobic, neutral |
| | neutral | M | |
| Cys, C | polar, hydrophobic, neutral | Asn, | polar, hydrophilic, neutral |
| | | N | |
| Asp, D | polar, hydrophilic, charged | Pro, P | hydrophobic, neutral |
| | (-) | | |
| Glu, E | polar, hydrophilic, charged | Gln, Q | polar, hydrophilic, neutral |
| | (-) | | |
| Phe, F | aromatic, hydrophobic, | Arg, R | polar, hydrophilic, |
| | neutral | | charged (+) |
| Gly, G | aliphatic, neutral | Ser, S | polar, hydrophilic, neutral |
| His, H | aromatic, polar, hydrophilic, | Thr, T | polar, hydrophilic, neutral |
| | charged (+) | | |
| Ile, I | aliphatic, hydrophobic, | Val, V | aliphatic, hydrophobic, |
| | neutral | | neutral |
| Lys, K | polar, hydrophilic, | Trp, | aromatic, hydrophobic, |
| | charged (+) | W | neutral |
| Leu, L | aliphatic, hydrophobic, | Tyr, Y | aromatic, polar, |
| | neutral | | hydrophobic |

Amino acids herein may be referred to by full name, three letter code or single letter code.

Preferred “variants” include those in which instead of the naturally occurring amino acid the amino acid which appears in the sequence is a structural analog thereof. Amino acids used in the sequences may also be derivatised or modified, e.g. labelled, providing the function of the antibody is not significantly adversely affected.

Derivatives and variants as described above may be prepared during synthesis of the antibody or by post-production modification, or when the antibody is in recombinant form using the known techniques of site-directed mutagenesis, random mutagenesis, or enzymatic cleavage and/or ligation of nucleic acids.

Suitable variants may be at least 70% homologous to a sequence disclosed herein, preferably at least 80% or 90% and more preferably at least 95%, 97% or 99% homologous thereto.

Preferably variants have an amino acid sequence which has more than 60%, or more than 70%, e.g. 75 or 80%, preferably more than 85%, e.g. more than 90 or 95% amino acid identity to a sequence as shown in the sequences disclosed herein (e.g. the VH or VL region sequences, or CDR sequences therein). This level of amino acid identity may be seen across the full length of the relevant SEQ ID NO sequence or over a part of the sequence, such as across 20, 30, 50, 75, 100, 150, 200 or more amino acids, depending on the size of the full-length polypeptide.

For example, variants of the above CDR sequences may comprise one, two three, four, five, six, seven, eight or more amino acid mutations relative to the reference sequence (such as a deletion, substitution and/or insertion of an amino acid).

The percent sequence identity between two polypeptides may be determined using suitable computer programs, for example the GAP program of the University of Wisconsin Genetic Computing Group and it will be appreciated that percent identity is calculated in relation to polypeptides whose sequences have been aligned optimally. Methods for determining sequence identity are known to those skilled in the art.

The alignment may alternatively be carried out using the Clustal W program (as described in Thompson et al., 1994, Nuc. Acid Res. 22:4673-4680, which is incorporated herein by reference).

The parameters used may be as follows:
Fast pairwise alignment parameters: K-tuple (word) size; 1, window size; 5, gap penalty; 3, number of top diagonals; 5. Scoring method: x percent.
Multiple alignment parameters: gap open penalty; 10, gap extension penalty; 0.05. Scoring matrix: BLOSUM.

Alternatively, the BESTFIT program may be used to determine local sequence alignments.

Also included within the scope of the invention are modified versions of antibodies and antigen-binding fragments thereof, e.g. modified by the covalent attachment of polyethylene glycol or other suitable polymers (see below).

Methods of generating antibodies and antibody fragments are well known in the art. For example, antibodies may be generated via any one of several methods which employ induction of in vivo production of antibody molecules, screening of immunoglobulin libraries (Orlandi et al, 1989. Proc. Natl. Acad. Sci. U.S.A. 86:3833-3837; Winter et al., 1991, Nature 349:293-299) or generation of monoclonal antibody molecules by cell lines in culture. These include, but are not limited to, the hybridoma technique, the human B-cell hybridoma technique, and the Epstein-Barr virus (EBV)-hybridoma technique (Kohler et al., 1975. Nature 256:4950497; Kozbor et al., 1985. J. Immunol. Methods 81:31-42; Cote et al., 1983. Proc. Natl. Acad. Sci. USA 80:2026-2030; Cole et al., 1984. Mol. Cell. Biol. 62:109-120).

Suitable monoclonal antibodies to selected antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982).

Likewise, antibody fragments can be obtained using methods well known in the art (see, for example, Harlow & Lane, 1988, “Antibodies: A Laboratory Manual”, Cold Spring Harbor Laboratory, New York). For example, antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Alternatively, antibody fragments can be obtained by pepsin or papain digestion of whole antibodies by conventional methods.

It will be appreciated by persons skilled in the art that for human therapy or diagnostics, human or humanised antibodies are preferably used. Humanised forms of non-human (e.g. murine) antibodies are genetically engineered chimeric antibodies or antibody fragments having preferably minimal-portions derived from non-human antibodies. Humanised antibodies include antibodies in which complementary determining regions of a human antibody (recipient antibody) are replaced by residues from a complementary determining region of a non-human species (donor antibody) such as mouse, rat of rabbit having the desired functionality. In some instances, Fv framework residues of the human antibody are replaced by corresponding non-human residues. Humanised antibodies may also comprise residues which are found neither in the recipient antibody nor in the imported complementarity determining region or framework sequences. In general, the humanised antibody will comprise substantially all of at least one, and typically two, variable domains, in which all or substantially all of the complementarity determining regions correspond to those of a non-human antibody and all, or substantially all, of the framework regions correspond to those of a relevant human consensus sequence. Humanised antibodies optimally also include at least a portion of an antibody constant region, such as an Fc region, typically derived from a human antibody (see, for example, Jones et al., 1986. Nature 321:522-525; Riechmann et al., 1988, Nature 332:323-329; Presta, 1992, Curr. Op. Struct. Biol. 2:593-596).

Methods for humanising non-human antibodies are well known in the art. Generally, the humanised antibody has one or more amino acid residues introduced into it from a source which is non-human. These non-human amino acid residues, often referred to as imported residues, are typically taken from an imported variable domain. Humanisation can be essentially performed as described (see, for example, Jones et al., 1986, Nature 321:522-525; Reichmann et al., 1988. Nature 332:323-327; Verhoeyen et al., 1988, Science 239:1534-15361; U.S. Pat. No. 4,816,567) by substituting human complementarity determining regions with corresponding rodent complementarity determining regions. Accordingly, such humanised antibodies are chimaeric antibodies, wherein substantially less than an intact human variable domain has been substituted by the corresponding sequence from a non-human species. In practice, humanised antibodies may be typically human antibodies in which some complementarity determining region residues and possibly some framework residues are substituted by residues from analogous sites in rodent antibodies.

Human antibodies can also be identified using various techniques known in the art, including phage display libraries (see, for example, Hoogenboom & Winter, 1991, J. Mol. Biol. 227:381; Marks et al., 1991, J. Mol. Biol. 222:581; Cole et al., 1985, In: Monoclonal antibodies and Cancer Therapy, Alan R. Liss, pp. 77; Boerner et al., 1991. J. Immunol. 147:86-95).

Once suitable antibodies are obtained, they may be tested for activity, for example by ELISA.

In some embodiments, the antigen-binding fragment thereof discussed herein is selected from: scFv, Fv, Fab, F(ab′)2, Fab-SH, dsFv, sdAb, di-scFvs, and Fcabs.

In some embodiments, the antibody or antigen-binding fragment thereof according to any of the preceding claims comprises a heavy chain constant region, or part thereof.

In some embodiments, the heavy chain constant region is of an immunoglobulin subtype selected from the group consisting of IgG1, IgG2, IgG3 and IgG4.

In some embodiments, the heavy chain constant region comprises a LALA mutation (e.g. Leu234Ala/Leu235Ala in IgG1) that reduces or eliminates binding to certain Fcγ receptors. In some other embodiments, the heavy chain constant region is modified so that it has a low level of fucosylation.

In some embodiments, the antibody or antigen-binding fragment thereof according to any of the preceding claims comprising a light chain constant region, or part thereof.

In some embodiments, the light chain constant region is of a kappa or lambda light chain.

In some preferred embodiments, the antibody is a whole antibody. By “whole antibody” we mean an antibody comprising a heavy and light chain variable and constant region.

In some embodiments, the antibody or antigen-binding fragment thereof is capable of inducing tumour specific immunity. This can be tested in vitro in T cell activation assays, e.g. by measuring. IL-2 and IFNγ production. Activation of effector T cells would imply that a tumour specific T cell response can be achieved in vivo. Further, an anti-tumour response in an in vivo model, such as a mouse model would imply that a successful immune response towards the tumour has been achieved.

The antibodies and antigen binding fragments of the invention may further comprise a moiety for increasing the in vivo half-life of the agent, such as but not limited to polyethylene glycol (PEG), human serum albumin, glycosylation groups, fatty acids and dextran. Such further moieties may be conjugated or otherwise combined with the binding moiety using methods well known in the art.

Likewise, it will be appreciated that the antibodies and antigen binding fragments thereof of the invention may further comprise a cytotoxic moiety. By “cytotoxic” we mean an agent which is toxic to cells, typically by killing the cells. The toxicity can lead to cell death by necrosis or apoptosis.

In some other embodiments, the antibodies and antigen binding fragments thereof may further comprise a cytostatic moiety. By “cytostatic” we mean an agent which inhibits or stops cell growth and/or multiplication.

In some embodiments, the cytotoxic or cytostatic moiety may comprise or consist of a radioisotope or a drug molecule.

A second aspect of the invention relates to an isolated nucleic acid molecule encoding an antibody or antigen-binding fragment thereof as disclosed herein, or a component polypeptide chain thereof.

The invention therefore relates to polynucleotides that encode all or part of an antibody or antigen binding fragment of the invention. The terms “nucleic acid molecule” and “polynucleotide” are used interchangeably herein and refer to a polymeric form of nucleotides of any length, either deoxyribonucleotides or ribonucleotides, or analogues thereof. Non-limiting examples of polynucleotides include a gene, a gene fragment, messenger RNA (mRNA), cDNA, recombinant polynucleotides, plasmids, vectors, isolated DNA of any sequence, isolated RNA of any sequence, nucleic acid probes, and primers. A polynucleotide of the invention may be provided in isolated or substantially isolated form. By substantially isolated, it is meant that there may be substantial, but not total, isolation of the polypeptide from any surrounding medium. The polynucleotides may be mixed with carriers or diluents which will not interfere with their intended use and still be regarded as substantially isolated.

A nucleic acid sequence which “encodes” a selected polypeptide is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, prokaryotic or eukaryotic mRNA, genomic sequences from viral or prokaryotic DNA or RNA, and even synthetic DNA sequences. A transcription termination sequence may be located 3′ to the coding sequence.

A suitable polynucleotide sequence may alternatively be a variant of a specific polynucleotide sequence, wherein the variant comprises mutations at one or more nucleotide position relative to the parent (unmutated) sequence. For example, a variant may be a substitution, deletion or addition variant. A variant polynucleotide may comprise 1, 2, 3, 4, 5, up to 10, up to 20, up to 30, up to 40, up to 50, up to 75 or more nucleic acid substitutions, insertions and/or deletions.

Suitable variants may be at least 70% homologous to a polynucleotide, preferably at least 80 or 90% and more preferably at least 95%, 97% or 99% homologous thereto. Preferably homology and identity at these levels is present at least with respect to the coding regions of the polynucleotides. Methods of measuring homology are well known in the art and it will be understood by those of skill in the art that in the present context, homology is calculated on the basis of nucleic acid identity. Such homology may exist over a region of at least 15, preferably at least 30, for instance at least 40, 60, 100, 200 or more contiguous nucleotides. Such homology may exist over the entire length of the unmodified polynucleotide sequence.

Methods of measuring polynucleotide homology or identity are known in the art. For example, the UWGCG Package provides the BESTFIT program which can be used to calculate homology (e.g. used on its default settings) (Devereux et al, 1984, Nucleic Acids Research 12:387-395; the disclosures of which are incorporated herein by reference).

The PILEUP and BLAST algorithms can also be used to calculate homology or line up sequences (typically on their default settings), for example as described in Altschul, 1993, J Mol Evol 36:290-300; Altschul et al, 1990, J Mol Biol 215:403-10, the disclosures of which are incorporated herein by reference).

Software for performing BLAST analysis is publicly available through the National Centre for Biotechnology Information. This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff & Henikoff, 1992, Proc. Natl. Acad. Sci. USA 89:10915-10919; the disclosures of which are incorporated herein by reference) alignments (B) of 50, expectation (E) of 10, M=5, N=4, and a comparison of both strands.

The BLAST algorithm performs a statistical analysis of the similarity between two sequences; see e.g. Karlin & Altschul, 1993, Proc. Natl. Acad. Sci. USA 90:5873-5787; the disclosures of which are incorporated herein by reference. One measure of similarity provided by the BLAST algorithm is the smallest sum probability (P(N)), which provides an indication of the probability by which a match between two nucleotide or amino acid sequences would occur by chance. For example, a sequence is considered similar to another sequence if the smallest sum probability in comparison of the first sequence to the second sequence is less than about 1, preferably less than about 0.1, more preferably less than about 0.01, and most preferably less than about 0.001.

The homologue may differ from a sequence in the relevant polynucleotide by less than 3, 5, 10, 15, 20 or more mutations (each of which may be a substitution, deletion or insertion). These mutations may be measured over a region of at least 30, for instance at least 40, 60 or 100 or more contiguous nucleotides of the homologue.

In one embodiment, a variant sequence may vary from the specific sequences given in the sequence listing by virtue of the redundancy in the genetic code. The DNA code has 4 primary nucleic acid residues (A, T, C and G) and uses these to “spell” three letter codons which represent the amino acids the proteins encoded in an organism's genes. The linear sequence of codons along the DNA molecule is translated into the linear sequence of amino acids in the protein(s) encoded by those genes. The code is highly degenerate, with 61 codons coding for the 20 natural amino acids and 3 codons representing “stop” signals. Thus, most amino acids are coded for by more than one codon—in fact several are coded for by four or more different codons. A variant polynucleotide of the invention may therefore encode the same polypeptide sequence as another polynucleotide of the invention, but may have a different nucleic acid sequence due to the use of different codons to encode the same amino acids.

A polypeptide of the invention may thus be produced from or delivered in the form of a polynucleotide which encodes and is capable of expressing it.

Polynucleotides of the invention can be synthesised according to methods well known in the art, as described by way of example in Green & Sambrook (2012, Molecular Cloning—a laboratory manual, 4^{th} edition; Cold Spring Harbor Press; the disclosures of which are incorporated herein by reference).

The nucleic acid molecules of the present invention may be provided in the form of an expression cassette which includes control sequences operably linked to the inserted sequence, thus allowing for expression of the polypeptide of the invention in vivo. These expression cassettes, in turn, are typically provided within vectors (e.g., plasmids or recombinant viral vectors). Such an expression cassette may be administered directly to a host subject. Alternatively, a vector comprising a polynucleotide of the invention may be administered to a host subject. Preferably the polynucleotide is prepared and/or administered using a genetic vector. A suitable vector may be any vector which is capable of carrying a sufficient amount of genetic information, and allowing expression of a polypeptide of the invention.

Therefore, a third aspect of the invention relates to a vector comprising a nucleic acid molecule as disclosed herein. In some embodiments, the vector is an expression vector.

The present invention thus includes expression vectors that comprise such polynucleotide sequences. Such expression vectors are routinely constructed in the art of molecular biology and may for example involve the use of plasmid DNA and appropriate initiators, promoters, enhancers and other elements, such as for example polyadenylation signals which may be necessary, and which are positioned in the correct orientation, in order to allow for expression of a peptide of the invention. Other suitable vectors would be apparent to persons skilled in the art.

A fourth aspect of the invention relates to a host cell comprising a nucleic acid molecule or a vector as disclosed herein. In some embodiments, the host cell is a bacterial cell, a mammalian cell, or a human cell.

Such cells include transient, or preferably stable higher eukaryotic cell lines, such as mammalian cells or insect cells, lower eukaryotic cells, such as yeast or prokaryotic cells such as bacterial cells. Particular examples of cells which may be modified by insertion of vectors or expression cassettes encoding for a polypeptide of the invention include mammalian HEK293T, CHO, HeLa, NS0 and COS cells. Preferably the cell line selected will be one which is not only stable, but also allows for mature glycosylation and cell surface expression of a polypeptide.

Such cell lines of the invention may be cultured using routine methods to produce a polypeptide of the invention, or may be used therapeutically or prophylactically to deliver antibodies of the invention to a subject. Alternatively, polynucleotides, expression cassettes or vectors of the invention may be administered to a cell from a subject ex vivo and the cell then returned to the body of the subject.

A fifth aspect of the invention relates to a pharmaceutical composition comprising an effective amount of an antibody or antigen-binding fragment thereof disclosed herein and a pharmaceutically-acceptable buffer, diluent, carrier or excipient. In some embodiments, the pharmaceutical composition is adapted for parenteral delivery or intravenous delivery.

Additional compounds may also be included in the compositions, including, chelating agents such as EDTA, citrate, EGTA or glutathione.

The pharmaceutical compositions may be prepared in a manner known in the art that is sufficiently storage stable and suitable for administration to humans and animals. For example, the pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the SLAMF6-binding activity of the agent of the invention. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazolelacetic acid, PIPES, SSC, SSPE, POPSO, TAPS, TABS, TAPSO and TES.

The term “diluent” is intended to mean an aqueous or non-aqueous solution with the purpose of diluting the agent in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the agent of the invention. The adjuvant may be one or more of zinc, copper or silver salts with different anions, for example, but not limited to fluoride, chloride, bromide, iodide, thiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrines, which are added to the composition, e.g. for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polysulphonate, polyethylene glycol/polyethylene oxide, polyethylene oxide/polypropylene oxide copolymers, polyvinylalcohol/polyvinylacetate of different degree of hydrolysis, and polyvinylpyrrolidone, all of different molecular weight, which are added to the composition, e.g. for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of the different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The agents of the invention may be formulated into any type of pharmaceutical composition known in the art to be suitable for the delivery thereof.

In one embodiment, the pharmaceutical compositions of the invention may be in the form of a liposome, in which the agent is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations can be found in for example U.S. Pat. No. 4,235,871, the disclosures of which are incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 0 213 303, the disclosures of which are incorporated herein by reference.

In a further embodiment, the pharmaceutical compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polyvinyl imidazole, polysulphonate, polyethylene glycol/polyethylene oxide, polyethylene oxide/polypropylene oxide copolymers, polyvinylalcohol/polyvinylacetate of degree different of hydrolysis, and polyvinylpyrrolidone are used for thickening of the solution containing the agent. The polymers may also comprise gelatin or collagen.

Alternatively, the agents may simply be dissolved in saline, water, polyethylene glycol, propylene glycol, ethanol or oils (such as safflower oil, corn oil, peanut oil, cottonseed oil or sesame oil), tragacanth gum, and/or various buffers.

It will be appreciated that the pharmaceutical compositions of the invention may include ions and a defined pH for potentiation of action of the active agent. Additionally, the compositions may be subjected to conventional pharmaceutical operations such as sterilisation and/or may contain conventional adjuvants such as preservatives, stabilisers, wetting agents, emulsifiers, buffers, fillers, etc.

The pharmaceutical compositions according to the invention may be administered via any suitable route known to those skilled in the art. Thus, possible routes of administration include parenteral (intravenous, subcutaneous, and intramuscular), topical, ocular, nasal, pulmonary, buccal, oral, parenteral, vaginal and rectal. Also, administration from implants is possible.

In one preferred embodiment, the pharmaceutical compositions are administered parenterally, for example, intravenously, intracerebroventricularly, intraarticularly, intra-arterially, intraperitoneally, intrathecally, intraventricularly, intrasternally, intracranially, intramuscularly or subcutaneously, or they may be administered by infusion techniques. They are conveniently used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well known to those skilled in the art.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Thus, the pharmaceutical compositions of the invention are particularly suitable for parenteral, e.g. intravenous, administration. In some preferred embodiments, the pharmaceutical compositions of the invention are administered intravenously.

Alternatively, the pharmaceutical compositions may be administered intranasally or by inhalation (for example, in the form of an aerosol spray presentation from a pressurised container, pump, spray or nebuliser with the use of a suitable propellant, such as dichlorodifluoromethane, trichlorofluoro-methane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas). In the case of a pressurised aerosol, the dosage unit may be determined by providing a valve to deliver a metered amount. The pressurised container, pump, spray or nebuliser may contain a solution or suspension of the active polypeptide, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of a compound of the invention and a suitable powder base such as lactose or starch.

The pharmaceutical compositions will be administered to a patient in a pharmaceutically effective dose. A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit or else several smaller dose units and also by multiple administrations of subdivided doses at specific intervals. Alternatively, the does may be provided as a continuous infusion over a prolonged period.

The compositions of the invention can be formulated at various concentrations, depending on the efficacy/toxicity of the compound being used. The skilled person will be aware of suitable techniques for determining formulations and dosages to be used in practice in administration to patients.

It will be appreciated by persons skilled in the art that the pharmaceutical compositions of the invention may be administered alone or in combination with other therapeutic agents used in the treatment of cancer, such as a chemotherapeutic agents. In particular, the therapeutic agent(s) may be ones known to be effective to the indication of interest.

A sixth aspect of the invention relates to a kit comprising an antibody or antigen-binding fragment thereof disclosed herein or a pharmaceutical composition as defined herein. In some embodiments, the kit comprises instructions for use.

A seventh aspect of the invention relates to an antibody or antigen-binding fragment thereof as disclosed herein for use in medicine.

An eighth aspect of the invention relates to an antibody or antigen-binding fragment thereof as disclosed herein, wherein the antibody or antigen-binding fragment is for use in treating cancer in an individual.

In some embodiments, the invention provides a method of treating a cancer in a subject, the method comprising administering a therapeutically effective amount of an antibody or antigen binding fragment disclosed herein or a pharmaceutical composition as defined herein.

In some embodiments, the cancer is a solid tumour. In some embodiments, the solid tumour is selected from: sarcoma, lymphoma, blastoma, melanoma, carcinoma, lung cancer, pancreatic cancer, breast cancer, gastrointestinal cancer, head and neck cancer, brain cancer, liver cancer, ovarian cancer, prostate cancer, and cervical cancer.

In some other embodiments, the cancer is a liquid tumour, e.g. a leukemia. In some embodiments, the myeloid malignancy/neoplasm is selected from myelodysplastic syndrome and/or myelodysplastic neoplasms (MDS), myeloproliferative neoplasms (MPN), mastocytosis, and myelodysplastic/myeloproliferative neoplasms, optionally wherein the myeloid malignancy and/or neoplasm is Acute Myeloid Leukemia (AML). In some preferred embodiments, the myeloid malignancy/neoplasm is AML.

In some embodiments, the invention provides a method of treating a myeloid malignancy and/or neoplasm in a subject, the method comprising administering a therapeutically effective amount of an antibody or antigen binding fragment disclosed herein or a pharmaceutical composition as defined herein.

Thus, in some embodiments, the invention provides methods for the treatment of cancers. By ‘treatment’ we include both therapeutic and prophylactic treatment of the patient. The term ‘prophylactic’ is used to encompass the use of a polypeptide or formulation described herein, which either prevents or reduces the likelihood of a myeloid malignancy and/or neoplasm in a patient or subject. We also include treatment of the patient to reduce the likelihood of relapse.

A ninth aspect of the invention relates to use of an antibody or antigen-binding fragment thereof as disclosed herein in the preparation of a medicament for inducing cell death and/or inhibiting the growth and/or proliferation of tumour cells. In some embodiments, this is achieved via the activation of T cells. In some embodiments, the tumour cells express SLAMF6.

A tenth aspect of the invention relates to use of an antibody or antigen-binding fragment thereof as disclosed herein in the preparation of a diagnostic agent for detecting cells associated with a tumour, wherein the cells express SLAMF6.

An eleventh aspect of the invention relates to use of an antibody or antigen-binding fragment thereof as disclosed herein for detecting cells associated with a tumour, wherein the cells express SLAMF6.

In some embodiments, the detection of cells associated with a tumour in an individual is used to diagnose said myeloid malignancy and/or neoplasm in an individual.

In some embodiments, the invention also provides an in vitro method for diagnosing or prognosing cancer in a subject using an antibody or antigen binding fragment as defined herein or a pharmaceutical composition as defined herein.

In one embodiment, the method is for determining whether an individual has or is at risk of developing a myeloid malignancy and/or neoplasm, the method comprising:
(a) providing a bone marrow or peripheral blood sample of haematopoietic cells from an individual to be tested;
(b) isolating a subpopulation of CD34⁺, CD38⁻ cells from the haematopoietic cells; and
(c) determining whether stem cells, contained within the CD34⁺, CD38⁻ cells, express the cell surface marker SLAMF6;
   wherein stem cells that exhibit the cell surface marker profile CD34⁺, CD38⁻ and SLAMF6⁺ are indicative of the individual having or developing a myeloid malignancy and/or neoplasm.

In some other embodiments, the method is for determining whether an individual has or is at risk of developing a solid tumour, the method comprising:
(a) providing a tissue sample of cells from an individual to be tested;
(b) determining whether cells express the cell surface marker SLAMF6;
   wherein cells that are SLAMF6⁺ are indicative of the individual having or developing a solid tumour and/or a solid tumour expressing SLAMF6. In some embodiments, this method is an immunohistochemistry method.

In some embodiments, the method also includes a step comprising quantification of levels of immune cells (such as B cells, T cells and/or NK cells), preferably wherein the immune cells express SLAMF6.

The provision of a sample is not necessarily to be construed as involving a surgical step. The provision of a sample could be a pre-isolated and stored frozen sample, for example.

A twelfth aspect of the invention relates to a method for inducing cell death and/or inhibiting the growth and/or proliferation of tumour cells in an individual in order to treat or prevent a cancer, comprising the step of administering to the individual an effective amount of an antibody or antigen binding fragment thereof, or a pharmaceutical composition as disclosed herein. In some embodiments, the tumour cells express SLAMF6.

A thirteenth aspect of the invention relates to a method for detecting cells associated with a tumour, comprising the step of administering to the individual an effective amount of an antibody or antigen binding fragment as defined herein, or a pharmaceutical composition as defined herein, wherein the cells express SLAMF6.

In some embodiments, the tumour is a cancer. In some embodiments, the tumour is a solid tumour. In some embodiments, the tumour is a liquid tumour. In some embodiments the solid tumour is selected from: sarcoma, lymphoma, blastoma, melanoma, carcinoma, lung cancer, pancreatic cancer, breast cancer, gastrointestinal cancer, head and neck cancer, brain cancer, liver cancer, ovarian cancer, prostate cancer, and cervical cancer.

In some preferred embodiments, the solid tumour is selected from the group consisting of: bowel cancer, colorectal cancer, esophageal cancer, stomach cancer, biliary tract cancer, kidney cancer, lung cancer, ovarian cancer, fallopian tube cancer, pancreatic cancer, or cancer of the soft tissues (e.g. sarcoma).

In some preferred embodiments, the solid tumour is colorectal cancer. In some preferred embodiments, the solid tumour is pancreatic cancer.

In some embodiments, the liquid tumour is a myeloid malignancy/neoplasm. In some embodiments, the myeloid malignancy/neoplasm is selected from myelodysplastic syndrome and/or myelodysplastic neoplasms (MDS), myeloproliferative neoplasms (MPN), mastocytosis, and myelodysplastic/myeloproliferative neoplasms, optionally wherein the myeloid malignancy and/or neoplasm is Acute Myeloid Leukemia (AML). In some preferred embodiments, the myeloid malignancy/neoplasm is AML.

In some embodiments the tumour is a pre-neoplastic condition such as clonal hematopoiesis of indeterminate potential (CHIP) or clonal cytopenia of unknown significance (CCUS).

In some embodiments, the method or use further comprises treating the individual for said cancer by administering or carrying out a treatment for the cancer.

By “treatment for cancer” we include any accepted treatment. Treatments may include, but are not limited to, the following: surgery, chemotherapy, radiotherapy, immunotherapy, CAR-T therapy, and bone marrow transplant.

Preferred, non-limiting examples which embody certain aspects of the invention will now be described, with reference to the following figures and examples:

### DESCRIPTION OF THE FIGURES

FIG. 1. Binding of generated SLAMF6 antibodies to SLAMF6-positive wildtype KG-1 cells (black), relative to an isotype-matched control antibody (grey). X-axis represents binding intensity. Y-axis represents normalized count.

FIG. 2. Binding of generated SLAMF6 antibodies to SLAMF6-positive wildtype KG-1 cells (black) and corresponding SLAMF6 knockout cells (grey). X-axis represents binding intensity. Y-axis represents normalized count.

FIG. 3. Induction of T cell-mediated killing (top) and T cell activation (bottom) by the SLAMF6 antibody LB-116 (A) and LB-031 (B) at indicated doses (black), relative to an isotype-matched control antibody (grey). T cell activation measured as the percentage of cells positive for surface expression of CD25.

FIG. 4. Induction of T cell-mediated killing of AML cells in vivo after treatment with LB-116 (A) and LB-031 (B). Left: AML engraftment in bone marrow. Middle: AML engraftment in spleen. Right: Spleen weights. Engraftment determined as the percentage of the total cell populations constituted by human AML cells in each tissue.

FIG. 5. Induction of T cell-mediated killing by different SLAMF6 antibodies at 1 ug/ml, relative to an isotype-matched control antibody. One representative experiment out of three, with different T cell donors.

FIG. 6. Induction of T cell-mediated killing by generated SLAMF6 antibodies at indicated concentrations, relative to an isotype-matched control antibody and to the SLAMF6 antibody 20F3 from Seagen Inc. One representative experiment out of three shown, all with T cells from different healthy donors.

FIG. 7. Binding epitopes in SLAMF6 as determined by HDX-MS for SLAMF6 antibodies that induce (LB-031, LB-116 and LB-20H10) and do not induce (Seagen 20F3) T cell activation. HDX-MS heatmaps with shading indicating the level of protection at each position at the indicated time points are shown along with the binding epitopes (black) in the SLAMF6 three-dimensional structure (grey).

FIG. 8. Gene expression of SLAMF6 in all cell lines in the 22Q4 dataset from the Cancer Cell Line Encyclopedia, grouped by cell lineage.

FIG. 9. Protein expression of SLAMF6 on the surface of the colorectal cancer cell lines C-99, DLD-1, HCC-56 and LS-513 (black), relative to an isotype-matched control antibody (grey).

FIG. 10. Protein expression of SLAMF6 on tumor cells in primary samples of colorectal cancer. Arrows indicate areas of notable SLAMF6 protein expression.

FIG. 11. Protein expression of SLAMF6 on the surface of the pancreatic cancer cell line MAPAC-HS-77 (black), relative to an isotype-matched control antibody (grey).

FIG. 12. Protein expression of SLAMF6 on tumor cells in primary samples of pancreatic cancer. Arrows indicate areas of notable SLAMF6 protein expression.

### EXAMPLES

### Example 1—Antibodies Display Specific Binding to SLAMF6

### Methods

SLAMF6 antibodies were generated by mouse immunizations (GenScript Biotech Corporation, Piscataway, NJ, USA) with the SLAMF6 extracellular domain (Innovagen AB, Lund, Sweden) or by phage-display scFv library screening (SciLifeLab DDD Platform, Solna, Sweden) using biotinylated SLAMF6 (Acro Biosystems #NTA-H82E6; Innovagen AB, Lund, Sweden). SLAMF6 knockout cells were generated using the Alt-R CRISPR-Cas9 System (Integrated DNA Technologies, Coralville, IA, USA), in which Cas9 protein, fluorochrome-conjugated tracrRNA and SLAMF6-specific crRNA were electroporated into the SLAMF6-positive human AML cell line KG-1 (DSMZ, Braunschweig, Germany), followed by fluorescence-activated cell sorting and expansion of SLAMF6 knockout cells. SLAMF6 knockout was verified by flow cytometry (BioLegend, San Diego, CA, USA #317208).

Antibody binding to SLAMF6 was determined by incubation of SLAMF6 wildtype and knockout cells with primary antibodies at 10 ug/ml followed by incubation with a secondary anti-human (Invitrogen, Waltham, MA, USA #H10120) or anti-mouse (BD Biosciences, Franklin Lakes, NJ, USA #550874) antibody and analysis by flow cytometry (BD LSR Fortessa II). KD values were determined by SPR using the Sierra SPR-16 biosensor (Bruker, Billerica, MA, USA).

### Results

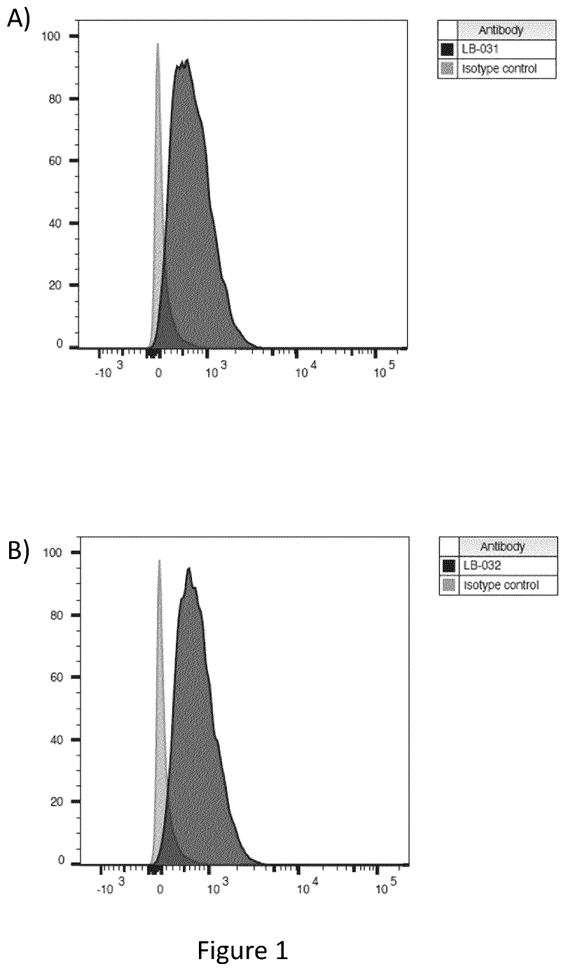

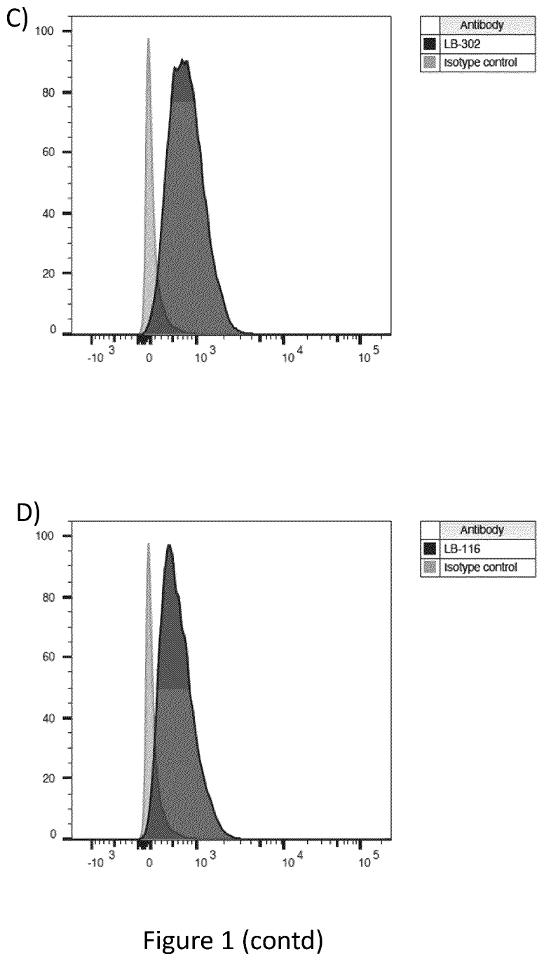

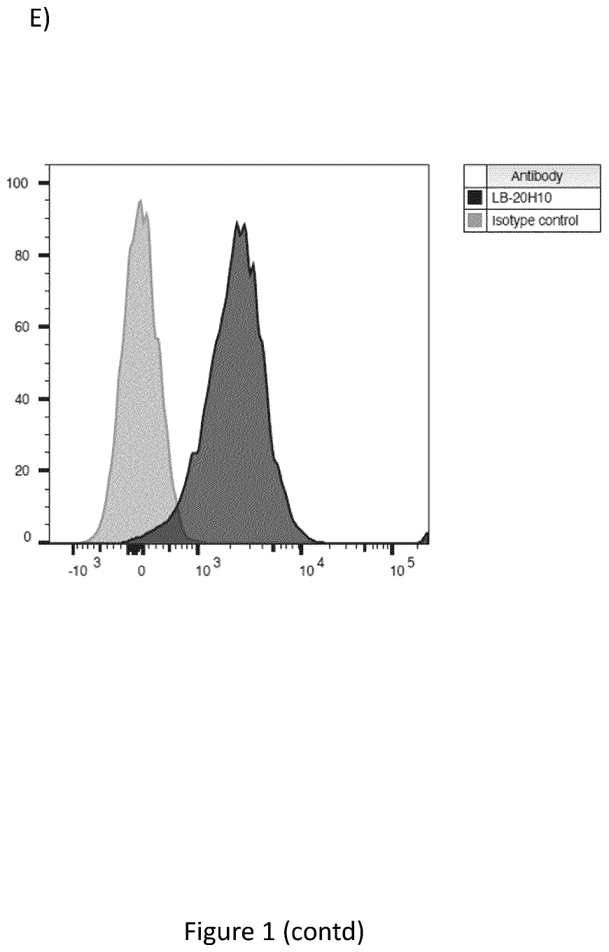

FIG. 1 shows that the antibodies LB-031, LB-032, LB-116, LB-302 and LB-20H10 bind to SLAMF6 on the surface of cancer cells.

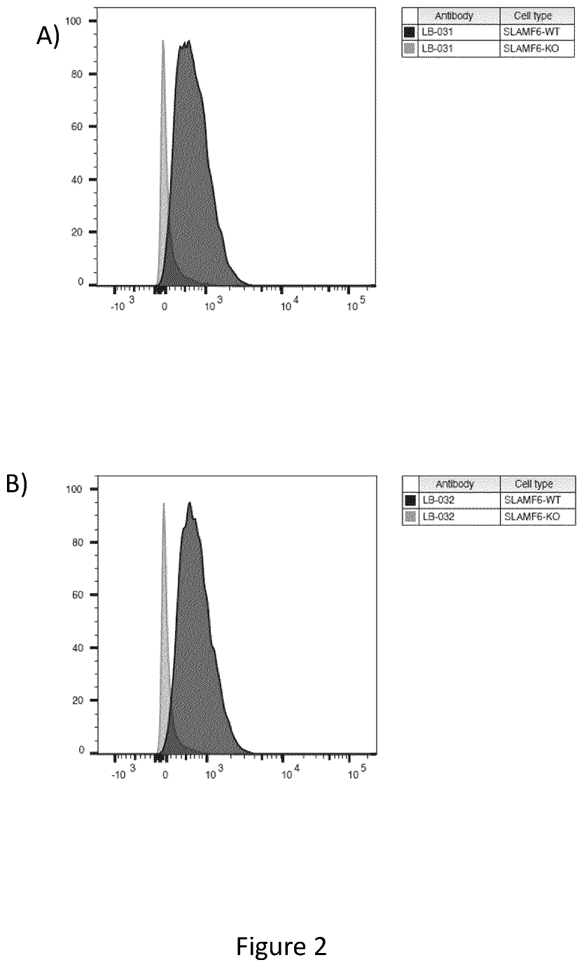

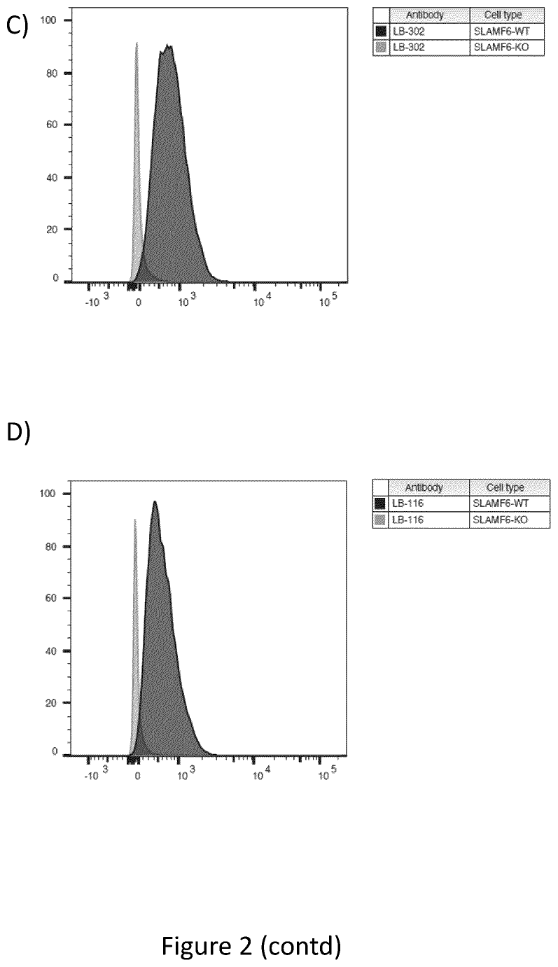

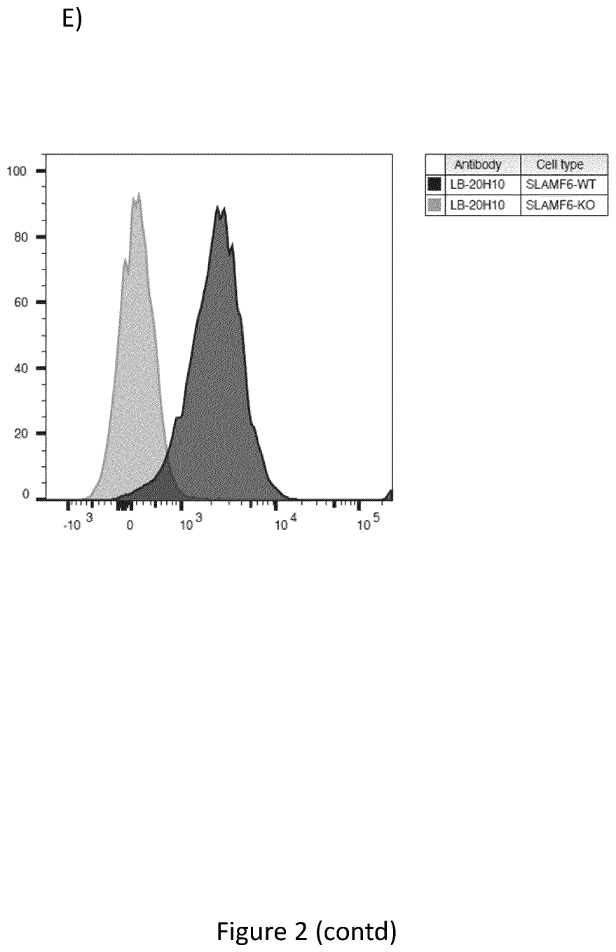

FIG. 2 shows that the binding of the antibodies LB-031, LB-032, LB-116, LB-302 and LB-20H10 is specific for SLAMF6, as the binding is ablated by knockout of SLAMF6 by CRISPR-Cas9.

Table 1 below shows the KD values determined for the antibodies:

TABLE 1

| Antibody | K_{D} |
|---|---|
| LB-031 | 3.8 × 10⁻¹⁰ |
| LB-032 | 1.2 × 10⁻⁸ |
| LB-116 | 6.0 × 10⁻⁹ |
| LB-302 | 1.6 × 10⁻⁸ |
| LB-20H10 | 4.1 × 10⁻⁹ |

KD values for the antibodies
LB-031, LB-032, LB-116,
LB-302, and 20H10.

### Conclusions

The antibodies LB-031, LB-032, LB-116, LB-302 and LB-20H10 bind specifically to the human SLAMF6 protein.

### Example 2

### SLAMF6 Antibody Induces T Cell Activation and Killing of AML Cells

### Methods

### In Vitro Treatment

The AML cell line HNT-34 was co-cultured with primary T cells isolated from peripheral blood from healthy donors (Miltenyi Biotec, Bergisch Gladbach, Germany #130-050-101) and either LB-031 or an isotype-matched control antibody (LALA-mutated hIgG1). After 72h, T cell activation and killing was analyzed by flow cytometry. T cell activation was determined by surface expression of CD25 (BioLegend #344816; BioLegend #302606) and T cell killing was determined by the number of remaining AML cells using CountBright absolute counting beads (Invitrogen #C36950; BioLegend #303416).

### In Vivo Treatment

Animal experiments were performed in the NOD.Cg-PrkdcscidIl2rgtm1Wjl/SzJ-SGM3 (NSG-S) mouse strain, a substrain of the NSG mouse overexpressing hGM-CSF, hIL-3 and hSCF (Jackson laboratory, Bar Harbor, ME, USA). Mice were sublethally irradiated (200 cGy) and transplanted with 5×10⁶ HNT-34 cells by tail vein injection. After 21 and 28 days, mice were transplanted with 1×10⁶ peripheral blood mononuclear cells from healthy donors, which had been isolated by Lymphoprep separation (Axis-Shield, Dundee, UK). Antibody treatment was administered by intraperitoneal injection 1 h and 72 h after each PBMC transplantation, at a dose of 2.0 mg/kg body weight. After 14 days of treatment, mice were sacrificed and the isolated bone marrow and spleen cells were stained with a panel of 7-AAD (BD Biosciences) and the following antibodies: CD3-PE/Cy7 (Biolegend #344816), CD33-BV421 (Biolegend #303416), CD34-AF488 (Biolegend #343518), CD45-APC (BD Biosciences #555485) and SLAMF6-PE (Biolegend #317208). Leukemic engraftment was determined by flow cytometry and defined as the percentage of hCD45⁺CD3⁻CD33⁺CD34⁺SLAMF6⁺ cells among the viable cells in each compartment.

### Results

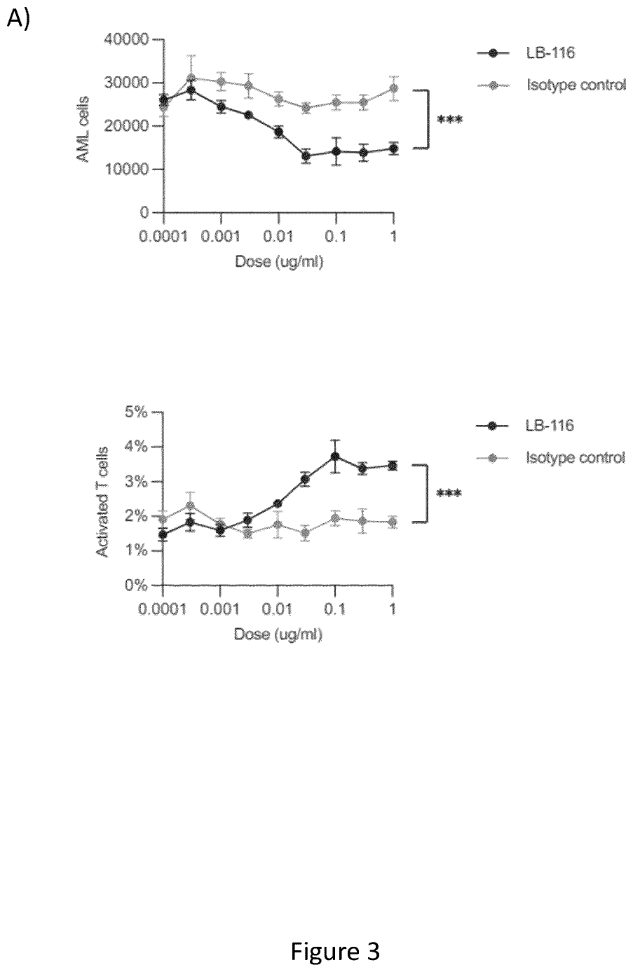

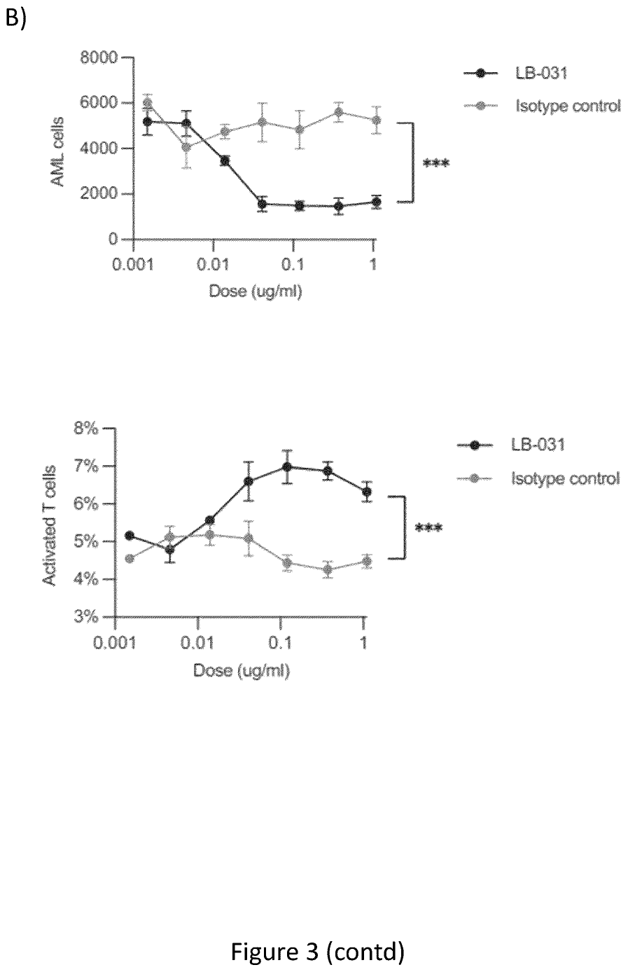

FIG. 3 shows that the LB-116 (FIG. 3A) and LB-031 (FIG. 3B) antibodies induce T cell activation and T cell-mediated killing of cancer cells in vitro.

FIG. 4 shows that the LB-116 (FIG. 4A) and LB-031 (FIG. 4B) antibodies induce T cell activation and T cell-mediated killing of cancer cells in vivo.

### Conclusions

The LB-031 and LB-116 antibodies induce T cell activation and T cell-mediated killing of cancer cells, both in vitro and in vivo.

### Example 3—Induction of T Cell Killing is Limited to a Subset SLAMF6 Antibodies with Specific Binding Properties

### Methods

All generated antibodies with verified specific binding to SLAMF6 were tested for induction of T cell-mediated killing at 1 ug/ml in three independent experiments with different T cell donors, as described above in Example 2. Antibodies with strong effects on T cell killing were subsequently tested at multiple concentrations and compared to the previously developed SLAMF6 antibody 20F3 from Seagen (Seagen Inc., Bothell, WA, USA; WO 2017/004330).

IC₅₀ values were determined based on three independent experiments with different T cell donors (Prism 9.5.1, GraphPad Software, San Diego, CA, USA). Epitope mapping was performed by hydrogen/deuterium exchange mass spectrometry (HDX-MS) using automated sample preparation on a LEAP H/D-X PAL™ platform (Trajan, Ringwood, Australia) interfaced to an LC-MS system, comprising an Ultimate 3000 micro-LC coupled to an Orbitrap Q Exactive Plus MS (ThermoFisher, Waltham, MA, USA). PEAKS Studio X Bioinformatics Solutions Inc. (BSI, Waterloo, Canada) was used for peptide identification after pepsin digestion of undeuterated samples. Peptides identified by PEAKS with a peptide score value of log P>25 and no modifications were used to generate a peptide list containing peptide sequence, charge state and retention time used for HDX data analysis and visualization with HDExaminer, version 3.1.1 (Sierra Analytics Inc., Modesto, US). Observed epitopes were mapped on AlphaFold model AF-Q96DU3-F1 (DeepMind, London, UK).

### Results

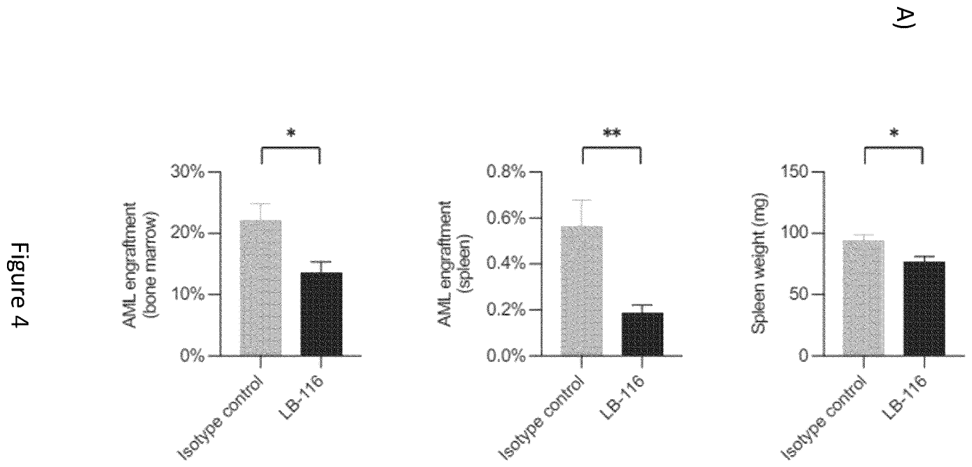

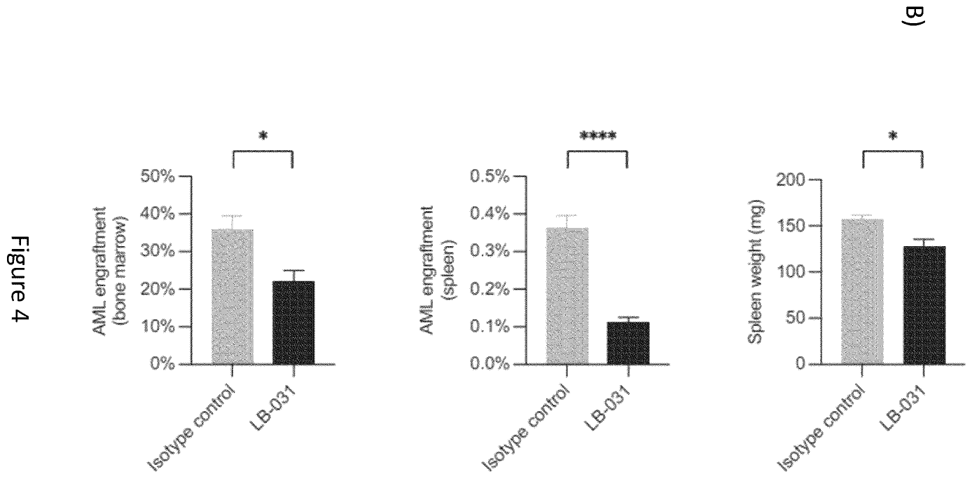

FIG. 5 shows that only a subset of the generated SLAMF6 antibodies (LB-20H10, LB-116, LB-031, LB-302, and LB-032) induce T cell-mediated killing compared to the control antibody. T cell mediated killing was surprising and was only induced for a subset of the anti-SLAMF6 antibodies generated.

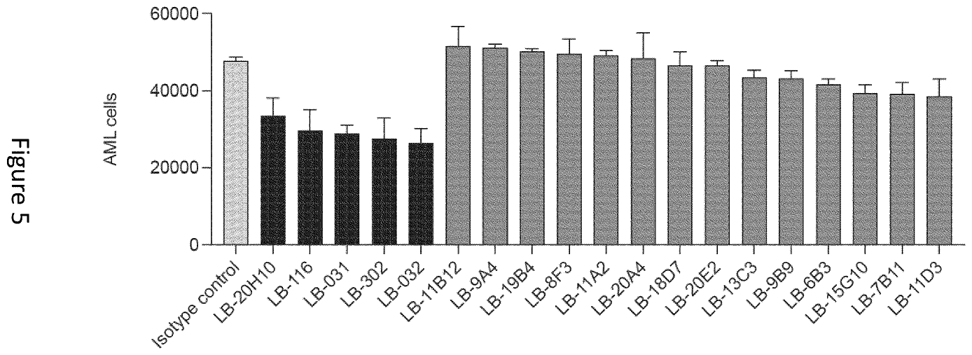

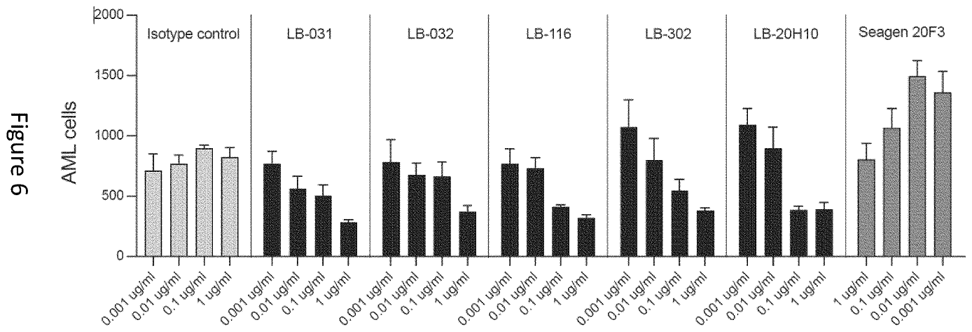

FIG. 6 shows that the generated SLAMF6 antibodies LB-031, LB-032, LB-116, LB-302 and LB-20H10 induce T cell-mediated killing, whereas the Seagen SLAMF6 antibody 20F3 does not.

Table 2 shows the IC₅₀ values for LB-031, LB-032, LB-116, LB-302 and LB-20H10:

TABLE 2

| Antibody | IC₅₀ |
|---|---|
| LB-031 | 0.035 ug/ml |
| LB-032 | 0.415 ug/ml |
| LB-116 | 0.023 ug/ml |
| LB-302 | 0.016 ug/ml |
| LB-20H10 | 0.026 ug/ml |

IC₅₀ values for the antibodies
LB-031, LB-032, LB-116,
LB-302 and LB-20H10.

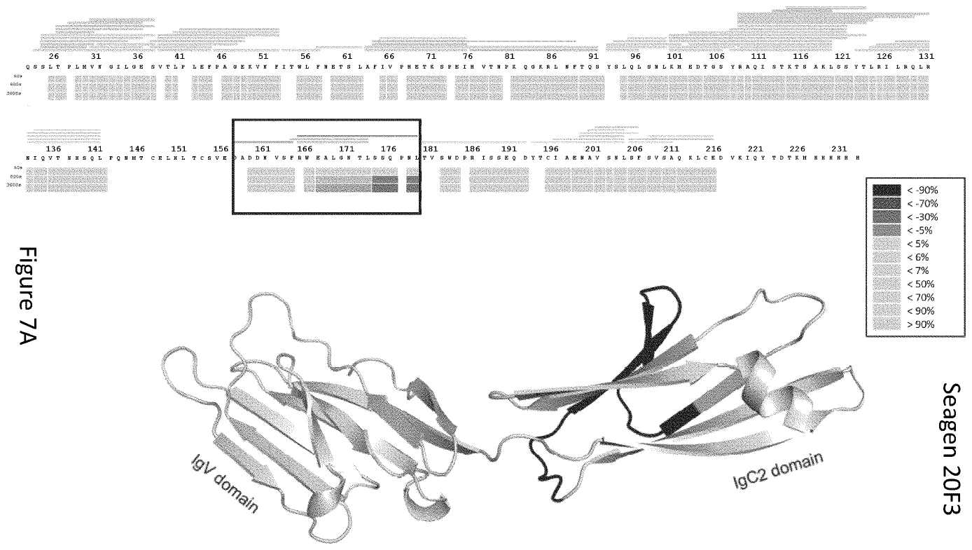

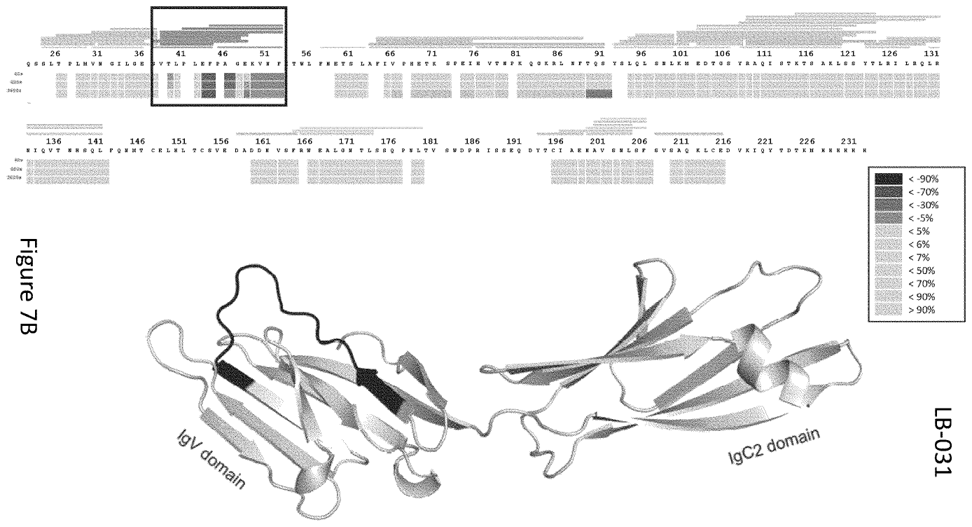

FIG. 7 and Table 3 show that the tested SLAMF6 antibodies that induce T cell killing (LB-031, LB-116 and LB-20H10) bind to the IgV domain of SLAMF6, whereas the Seagen SLAMF6 antibody that does not induce T cell-mediated killing (20F3) binds to the IgC2 domain of SLAMF6.

TABLE 3
Binding epitopes in SLAMF6 for the antibodies LB-031, LB-116 and LB-20H10
that induce T cell killing and the Seagen 20F3 antibody that does not.
“SLAMF6 domain” denotes the domain in the SLAMF6 protein to which each
antibody binds. “Epitope” denotes the binding epitope for each antibody,
as amino acid numbers in SLAMF6 protein. “Sequence” denotes the amino acid
sequence of the binding epitope in the SLAMF6 protein.

| | T cell | SLAMF6 | | |
|---|---|---|---|---|
| Antibody | killing | domain | Epitope | Sequence |
| Seagen | No | IgC2 | 157-179 | DADDNVSFRWEALGNTLSSQPNL (SEQ |
| 20F3 | | | | ID NO. 101) |
| LB-031 | Yes | IgV | 37-52 | SVTLPLEFPAGEKVNF (SEQ ID NO. 102) |
| LB-116 | Yes | IgV | 63-91 | AFIVPHETKSPEIHVTNPKQGKRLNFTQS |
| | | | | (SEQ ID NO. 103) |
| LB-20H10 | Yes | IgV | 100-123 | KMEDTGSYRAQISTKTSAKLSSYT (SEQ |
| | | | | ID NO. 104) |

### Conclusions

Antibodies that bind to the IgV domain of the SLAMF6 protein induce T cell-mediated killing of cancer cells, whereas antibodies that bind to the IgC2 domain do not. The generated antibodies LB-031, LB-032, LB-116, LB-302 and LB-20H10 induce T cell-mediated killing, while the existing SLAMF6 antibody 20F3 from Seagen does not.

Therefore, the antibodies LB-031, LB-032, LB-116, LB-302 and LB-20H10 are superior to the 20F3 antibody, as they induce T-cell mediated killing of cancer cells, meaning their therapeutic effect is superior.

### Example 4—SLAMF6 is Aberrantly Expressed in Solid Tumors from Multiple Tissues

### Methods

SLAMF6 gene expression was analyzed across all cell lines in the publicly available 22Q4 dataset in the Cancer Cell Line Encyclopedia (https://depmap.org; downloaded on Feb. 28, 2023).

### Results

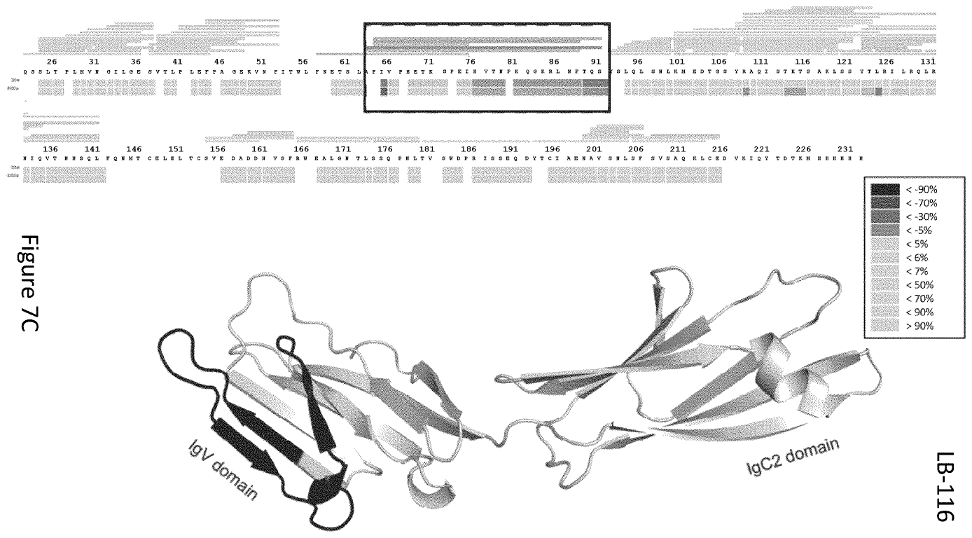

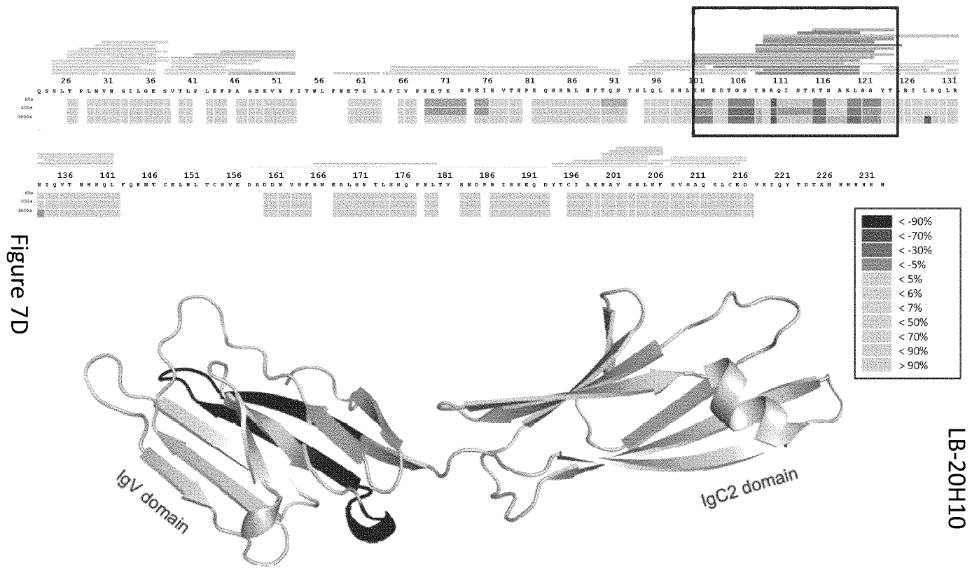

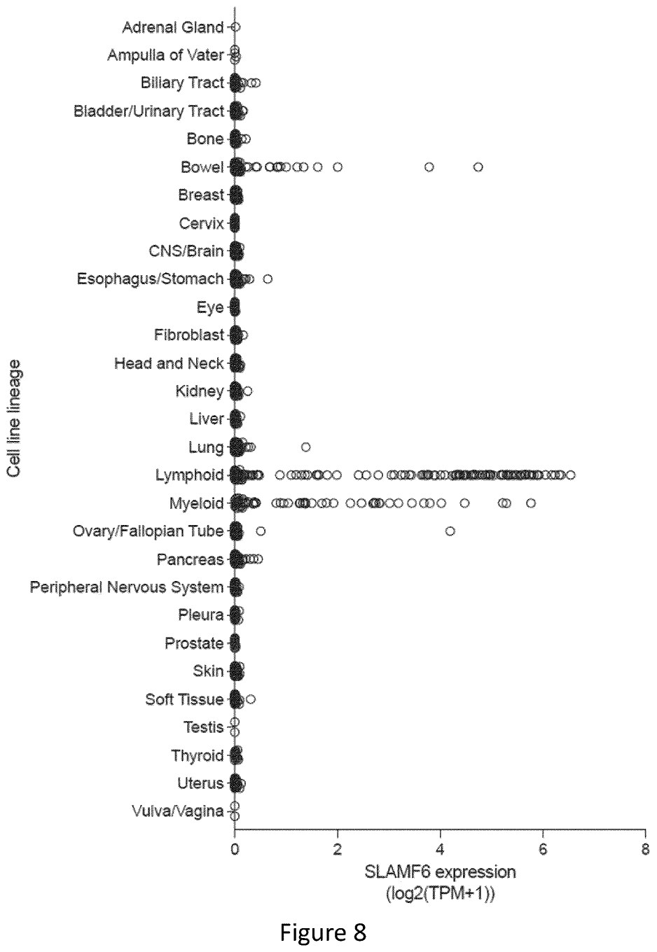

FIG. 8 shows that SLAMF6 is aberrantly expressed in cancer cell lines from solid tumors from multiple tissues.

### Conclusions

SLAMF6 is aberrantly expressed in solid tumors from multiple tissues, and therefore represents a potential target for antibody therapy in solid tumor patients.

### Example 5—SLAMF6 is Aberrantly Expressed in Colorectal Cancer

### Methods

SLAMF6 surface protein expression on colorectal cancer cells was detected by flow cytometry (BioLegend #317208) on the cell lines C-99 (Merck, Darmstadt, Germany), DLD-1 (ATCC, Manassas, VA, USA), HCC-56 (provided by the laboratory of Prof. Walter Bodmer) and LS-513 (ATCC), which were positive for SLAMF6 gene expression in the 22Q4 dataset. SLAMF6 protein expression on primary samples of colorectal cancer was detected by immunohistochemistry (LSBio, Seattle, WA, USA #LS-B15877-50).

### Results

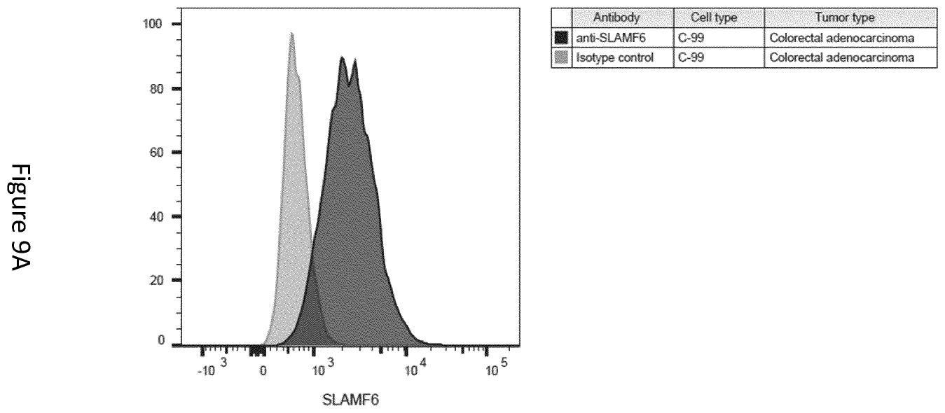

FIG. 9 shows that SLAMF6 protein is expressed on the surface of colorectal cancer cell lines.

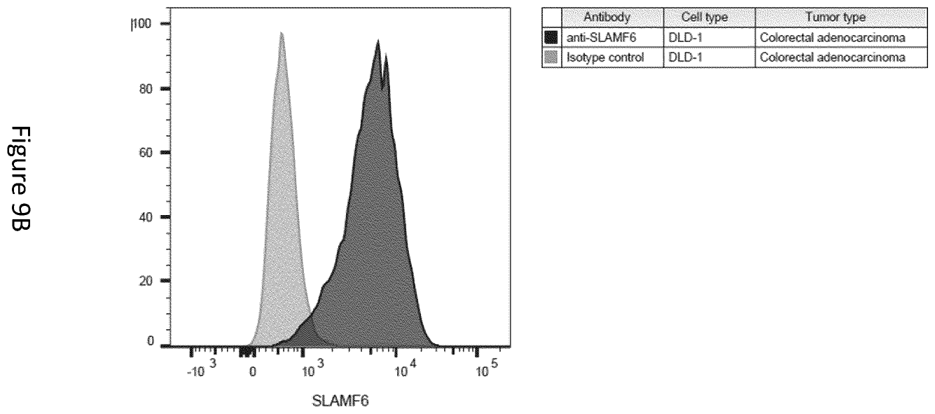

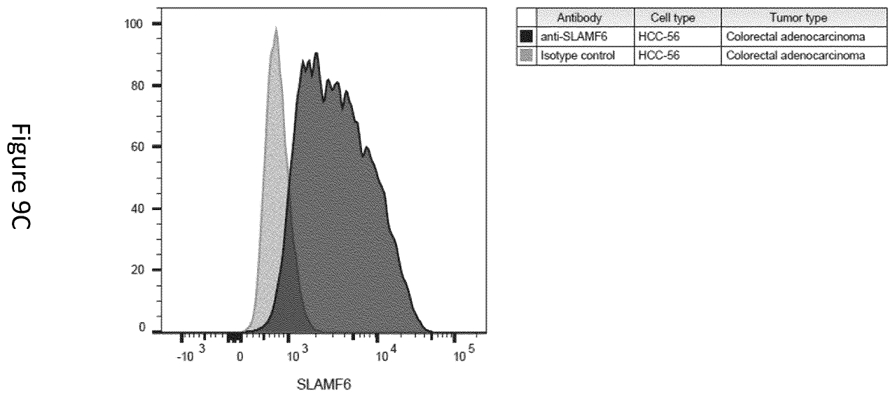

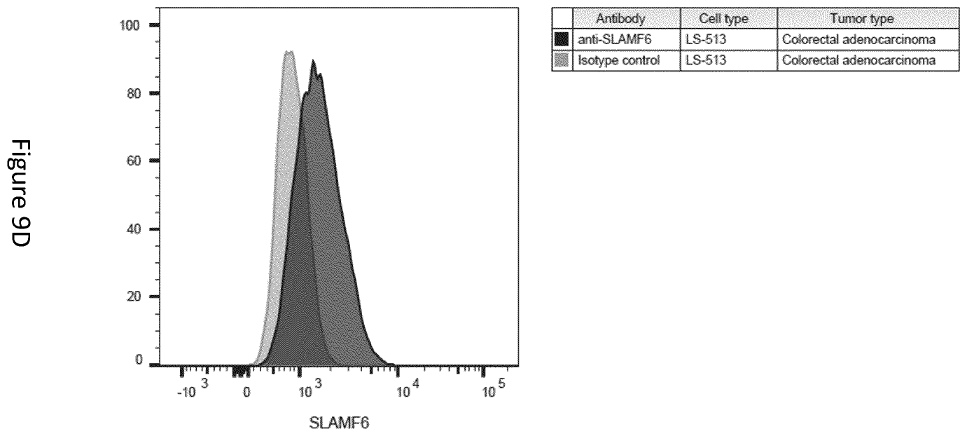

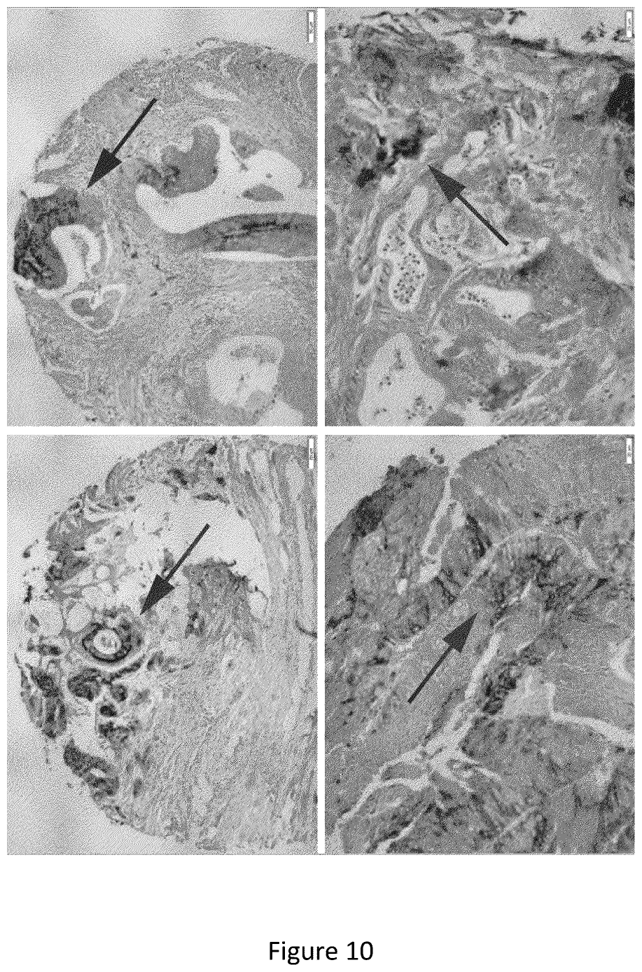

FIG. 10 shows that SLAMF6 protein is expressed on tumor cells in primary material from cases of colorectal cancer.

### Conclusions

SLAMF6 protein is aberrantly expressed on the surface of colorectal cancer cells, and therefore represents a potential target for antibody therapy in colorectal cancer patients.

### Example 6—SLAMF6 is Aberrantly Expressed in Pancreatic Cancer

### Methods

SLAMF6 surface protein expression on colorectal cancer cells was detected by flow cytometry (BioLegend #317208) on the cell line MAPAC-HS-77 (DSMZ), which was positive for SLAMF6 gene expression in the 22Q4 dataset. SLAMF6 protein expression on primary samples of pancreatic cancer was detected by immunohistochemistry (LSBio #LS-B15877-50).

### Results

FIG. 11 shows that SLAMF6 protein is expressed on the surface of a pancreatic cancer cell line.

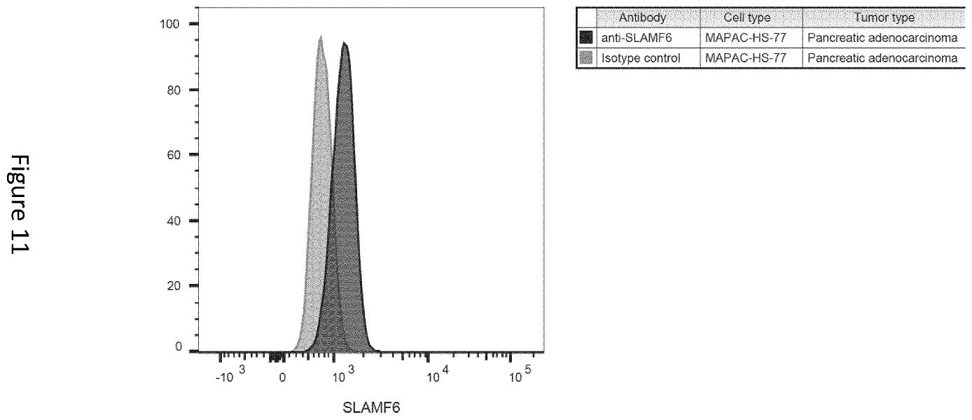

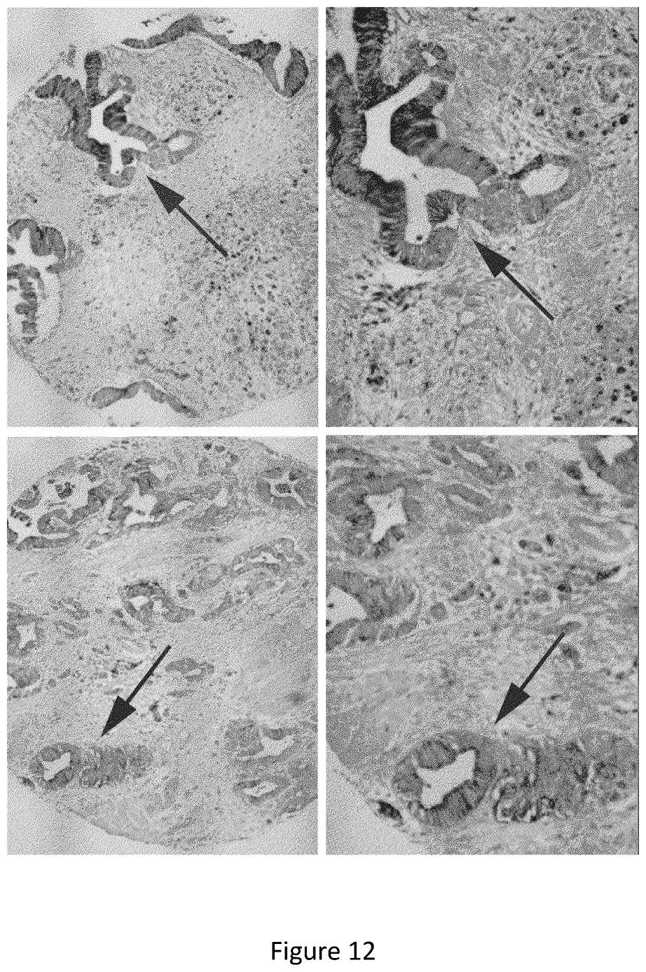

FIG. 12 shows that SLAMF6 protein is expressed on tumor cells in primary material from cases of pancreatic cancer.

### Conclusions

SLAMF6 protein is aberrantly expressed on the surface of pancreatic cancer cells, and therefore represents a potential target for antibody therapy in pancreatic cancer patients.

Sequences

| SEQ | Description | |
|---|---|---|
| ID | of | |
| No. | sequence | Sequence |
| 1 | LB-031 VH | EVQLLESGGGLVQPGGSLRLSCAASGFTFGSYYMGWVRQAPGKGLEWVSY |
| | | ISGYSGYTYYADSVKGRFTISRDNSKNTLYLQMNSLRAEDTAVYYCARFS |
| | | SPAGYPRMDYWGQGTLVTVSS |
| 2 | LB-031 VL | DIQMTQSPSSLSASVGDHVTITCRASQSISSYLNWYQQKPGKAPKLLIYA |
| | | ASSLQSGVPSRFSGSGSGTDFTLTISSLQPEDFATYYCQQYYTLPTFGQG |
| | | TKLEIK |
| 3 | LB-032 VH | EVQLLESGGGLVQPGGSLRLSCAASGFTFGSYGMSWVRQAPGKGLEWVSY |
| | | ISYSGSYTYYADSVKGRFTISRDNSKNTLYLQMNSLRAEDTAVYYCARGA |
| | | DYYSVLDYWGQGTLVTVSS |
| 4 | LB-032 VL | DIQMTQSPSSLSASVGDHVTITCRASQSISSYLNWYQQKPGKAPKLLIYA |
| | | ASSLQSGVPSRFSGSGSGTDFTLTISSLQPEDFATYYCQQFSTSPPSTFG |
| | | QGTKLEIK |
| 5 | LB-302 VH | EVQLLESGGGLVQPGGSLRLSCAASGFTFGGYGMSWVRQAPGKGLEWVSY |
| | | IGYGGSYTYYADSVKGRFTISRDNSKNTLYLQMNSLRAEDTAVYYCARDG |
| | | AGSYGFFDYWGQGTLVTVSS |
| 6 | LB-302 VL | DIQMTQSPSSLSASVGDHVTITCRASQSISSYLNWYQQKPGKAPKLLIYA |
| | | ASSLQSGVPSRFSGSGSGTDFTLTISSLQPEDFATYYCQQFYTNPLLTFG |
| | | QGTKLEIK |
| 7 | LB-116 VH | EVQLLESGGGLVQPGGSLRLSCAASGFTFSYSYMYWVRQAPGKGLEWVSS |
| | | ISSSGGSTSYADSVKGRFTISRDNSKNTLYLQMNSLRAEDTAVYYCARPN |
| | | VDSYMDYWGQGTLVTVSS |
| 8 | LB-116 VL | DIQMTQSPSSLSASVGDHVTITCRASQSISSYLNWYQQKPGKAPKLLIYA |
| | | ASSLQSGVPSRFSGSGSGTDFTLTISSLQPEDFATYYCQQSYIYLPFTFG |
| | | QGTKLEIK |
| 9 | LB-20H10 VH | QVQLQQSGAELVRPGTSVKMSCKAAGYTFTNYWIGWVKQRPGHGLEWIGD |
| | | IYPGGGYTHHNEKFKGKATLTADTSSSTAYMQLSSLTSEDSAIYYCARSV |
| | | LPGFAYWGQGTLVTVSA |
| 10 | LB-20H10 VL | DIVMTQSHKFMSTSVGDRVSITCKASQDVNTAVAWCQQKPGQSPKLLIYS |
| | | ASYRFTGVPDRFTGSGSGTDFTFTISSVQAEDLAVYFCQQHYSTPYTFGG |
| | | GTKLEIK |
| 11 | LB-031 CDR-H1 | GFTFGSY |
| | (Chothia) | |
| 12 | LB-031 CDR-H1 | SYYMG |
| | (Kabat) | |
| 13 | LB-031 CDR-H1 | GFTFGSYY |
| | (IMGT) | |
| 14 | LB-031 CDR-H2 | SGYSGY |
| | (Chothia) | |
| 15 | LB-031 CDR-H2 | YISGYSGYTYYADSVKG |
| | (Kabat) | |
| 16 | LB-031 CDR-H2 | ISGYSGYT |
| | (IMGT) | |
| 17 | LB-031 CDR-H3 | FSSPAGYPRMDY |
| | (Chothia) | |
| 18 | LB-031 CDR-H3 | FSSPAGYPRMDY |
| | (Kabat) | |
| 19 | LB-031 CDR-H3 | ARFSSPAGYPRMDY |
| | (IMGT) | |
| 20 | LB-031 CDR-L1 | RASQSISSYLN |
| | (Chothia) | |
| 21 | LB-031 CDR-L1 | RASQSISSYLN |
| | (Kabat) | |
| 22 | LB-031 CDR-L1 | QSISSY |
| | (IMGT) | |
| 23 | LB-031 CDR-L2 | AASSLQS |
| | (Chothia) | |
| 24 | LB-031 CDR-L2 | AASSLQS |
| | (Kabat) | |
| 25 | LB-031 CDR-L2 | AA |
| | (IMGT) | |
| 26 | LB-031 CDR-L3 | QQYYTLPT |
| | (Chothia) | |
| 27 | LB-031 CDR-L3 | QQYYTLPT |
| | (Kabat) | |
| 28 | LB-031 CDR-L3 | QQYYTLPT |
| | (IMGT) | |
| 29 | LB-032 CDR-H1 | GFTFGSY |
| | (Chothia) | |
| 30 | LB-032 CDR-H1 | SYGMS |
| | (Kabat) | |
| 31 | LB-032 CDR-H1 | GFTFGSYG |
| | (IMGT) | |
| 32 | LB-032 CDR-H2 | SYSGSY |
| | (Chothia) | |
| 33 | LB-032 CDR-H2 | YISYSGSYTYYADSVKG |
| | (Kabat) | |
| 34 | LB-032 CDR-H2 | ISYSGSYT |
| | (IMGT) | |
| 35 | LB-032 CDR-H3 | GADYYSVLDY |
| | (Chothia) | |
| 36 | LB-032 CDR-H3 | GADYYSVLDY |
| | (Kabat) | |
| 37 | LB-032 CDR-H3 | ARGADYYSVLDY |
| | (IMGT) | |
| 38 | LB-032 CDR-L1 | RASQSISSYLN |
| | (Chothia) | |
| 39 | LB-032 CDR-L1 | RASQSISSYLN |
| | (Kabat) | |
| 40 | LB-032 CDR-L1 | QSISSY |
| | (IMGT) | |
| 41 | LB-032 CDR-L2 | AASSLQS |
| | (Chothia) | |
| 42 | LB-032 CDR-L2 | AASSLQS |
| | (Kabat) | |
| 43 | LB-032 CDR-L2 | AA |
| | (IMGT) | |
| 44 | LB-032 CDR-L3 | QQFSTSPPST |
| | (Chothia) | |
| 45 | LB-032 CDR-L3 | QQFSTSPPST |
| | (Kabat) | |
| 46 | LB-032 CDR-L3 | QQFSTSPPST |
| | (IMGT) | |
| 47 | LB-302 CDR-H1 | GFTFGGY |
| | (Chothia) | |
| 48 | LB-302 CDR-H1 | GYGMS |
| | (Kabat) | |
| 49 | LB-302 CDR-H1 | GFTFGGYG |
| | (IMGT) | |
| 50 | LB-302 CDR-H2 | GYGGSY |
| | (Chothia) | |
| 51 | LB-302 CDR-H2 | YIGYGGSYTYYADSVKG |
| | (Kabat) | |
| 52 | LB-302 CDR-H2 | IGYGGSYT |
| | (IMGT) | |
| 53 | LB-302 CDR-H3 | DGAGSYGFFDY |
| | (Chothia) | |
| 54 | LB-302 CDR-H3 | DGAGSYGFFDY |
| | (Kabat) | |
| 55 | LB-302 CDR-H3 | ARDGAGSYGFFDY |
| | (IMGT) | |
| 56 | LB-302 CDR-L1 | RASQSISSYLN |
| | (Chothia) | |
| 57 | LB-302 CDR-L1 | RASQSISSYLN |
| | (Kabat) | |
| 58 | LB-302 CDR-L1 | QSISSY |
| | (IMGT) | |
| 59 | LB-302 CDR-L2 | AASSLQS |
| | (Chothia) | |
| 60 | LB-302 CDR-L2 | AASSLQS |
| | (Kabat) | |
| 61 | LB-302 CDR-L2 | AA |
| | (IMGT) | |
| 62 | LB-302 CDR-L3 | QQFYTNPLLT |
| | (Chothia) | |
| 63 | LB-302 CDR-L3 | QQFYTNPLLT |
| | (Kabat) | |
| 64 | LB-302 CDR-L3 | QQFYTNPLLT |
| | (IMGT) | |
| 65 | LB-116 CDR-H1 | GFTFSYS |
| | (Chothia) | |
| 66 | LB-116 CDR-H1 | YSYMY |
| | (Kabat) | |
| 67 | LB-116 CDR-H1 | GFTFSYSY |
| | (IMGT) | |
| 68 | LB-116 CDR-H2 | SSSGGS |
| | (Chothia) | |
| 69 | LB-116 CDR-H2 | SISSSGGSTSYADSVKG |
| | (Kabat) | |
| 70 | LB-116 CDR-H2 | ISSSGGST |
| | (IMGT) | |
| 71 | LB-116 CDR-H3 | PNVDSYMDY |
| | (Chothia) | |
| 72 | LB-116 CDR-H3 | PNVDSYMDY |
| | (Kabat) | |
| 73 | LB-116 CDR-H3 | ARPNVDSYMDY |
| | (IMGT) | |
| 74 | LB-116 CDR-L1 | RASQSISSYLN |
| | (Chothia) | |
| 75 | LB-116 CDR-L1 | RASQSISSYLN |
| | (Kabat) | |
| 76 | LB-116 CDR-L1 | QSISSY |
| | (IMGT) | |
| 77 | LB-116 CDR-L2 | AASSLQS |
| | (Chothia) | |
| 78 | LB-116 CDR-L2 | AASSLQS |
| | (Kabat) | |
| 79 | LB-116 CDR-L2 | AA |
| | (IMGT) | |
| 80 | LB-116 CDR-L3 | QQSYIYLPFT |
| | (Chothia) | |
| 81 | LB-116 CDR-L3 | QQSYIYLPFT |
| | (Kabat) | |
| 82 | LB-116 CDR-L3 | QQSYIYLPFT |
| | (IMGT) | |
| 83 | LB-20H10 CDR- | GYTFTNY |
| | H1 (Chothia) | |
| 84 | LB-20H10 CDR- | NYWIG |
| | H1 (Kabat) | |
| 85 | LB-20H10 CDR- | GYTFTNYW |
| | H1 (IMGT) | |
| 86 | LB-20H10 CDR- | YPGGGY |
| | H2 (Chothia) | |
| 87 | LB-20H10 CDR- | DIYPGGGYTHHNEKFKG |
| | H2 (Kabat) | |
| 88 | LB-20H10 CDR- | IYPGGGYT |
| | H2 (IMGT) | |
| 89 | LB-20H10 CDR- | SVLPGFAY |
| | H3 (Chothia) | |
| 90 | LB-20H10 CDR- | SVLPGFAY |
| | H3 (Kabat) | |
| 91 | LB-20H10 CDR- | ARSVLPGFAY |
| | H3 (IMGT) | |
| 92 | LB-20H10 CDR- | KASQDVNTAVA |
| | L1 (Chothia) | |
| 93 | LB-20H10 CDR- | KASQDVNTAVA |
| | L1 (Kabat) | |
| 94 | LB-20H10 CDR- | QDVNTA |
| | L1 (IMGT) | |
| 95 | LB-20H10 CDR- | SASYRFT |
| | L2 (Chothia) | |
| 96 | LB-20H10 CDR- | SASYRFT |
| | L2 (Kabat) | |
| 97 | LB-20H10 CDR- | SA |
| | L2 (IMGT) | |
| 98 | LB-20H10 CDR- | QQHYSTPYT |
| | L3 (Chothia) | |
| 99 | LB-20H10 CDR- | QQHYSTPYT |
| | L3 (Kabat) | |
| 100 | LB-20H10 CDR- | QQHYSTPYT |
| | L3 (IMGT) | |

1. An antibody or an antigen-binding fragment thereof that binds specifically to Signaling Lymphocytic Activating Molecule Family Member 6 (SLAMF6), wherein the antibody or antigen-binding fragment thereof is capable of inducing immune cell mediated killing of tumour cells.

2. An antibody or antigen-binding fragment thereof of claim 1, wherein the antibody or antigen-binding fragment thereof is capable of binding to and activating immune cells, optionally wherein the immune cells are T cells, B cells and/or NK cells, optionally wherein the immune cells are T cells and activating said T cells enhances T cell mediated killing.

3. An antibody or antigen-binding fragment thereof of any of the preceding claims, wherein the antibody or antigen-binding fragment thereof is capable of binding directly to tumour cells and mediating direct tumour cell killing.

4. An antibody or antigen-binding fragment thereof of any of the preceding claims comprising a heavy chain variable region comprising one or more of the CDRs of SEQ ID NOs 7, 1, 3, 5 or 9.

5. An antibody or antigen-binding fragment thereof of any of the preceding claims comprising a heavy chain variable region comprising the following CDRs:
a) SEQ ID NOs 65, 66, or 67 or an amino acid sequence having at least 60% sequence identity therewith, for example at least 70%, 80%, or 90% sequence identity; and/or
b) SEQ ID NOs 68, 69, or 70 or an amino acid sequence having at least 60% sequence identity therewith, for example at least 70%, 80%, or 90% sequence identity; and/or
c) SEQ ID NOs 71, 72, or 73 or an amino acid sequence having at least 60% sequence identity therewith, for example at least 70%, 80%, or 90% sequence identity; or
a) SEQ ID NOs 11, 12, or 13 or an amino acid sequence having at least 60% sequence identity therewith, for example at least 70%, 80%, or 90% sequence identity; and/or
b) SEQ ID NOs 14, 15, or 16 or an amino acid sequence having at least 60% sequence identity therewith, for example at least 70%, 80%, or 90% sequence identity; and/or
c) SEQ ID NOs 17, 18, or 19 or an amino acid sequence having at least 60% sequence identity therewith, for example at least 70%, 80%, or 90% sequence identity; or
d) SEQ ID NOs 29, 30 or 31, or an amino acid sequence having at least 60% sequence identity therewith, for example at least 70%, 80%, or 90% sequence identity; and/or
e) SEQ ID NOs 32, 33, or 34 or an amino acid sequence having at least 60% sequence identity therewith, for example at least 70%, 80%, or 90% sequence identity; and/or
f) SEQ ID NOs 35, 36, or 37 or an amino acid sequence having at least 60% sequence identity therewith, for example at least 70%, 80%, or 90% sequence identity; or
g) SEQ ID NOs 47, 48, or 49 or an amino acid sequence having at least 60% sequence identity therewith, for example at least 70%, 80%, or 90% sequence identity; and/or
h) SEQ ID NOs 50, 51, or 52 or an amino acid sequence having at least 60% sequence identity therewith, for example at least 70%, 80%, or 90% sequence identity; and/or
i) SEQ ID NOs 53, 54, or 55 or an amino acid sequence having at least 60% sequence identity therewith, for example at least 70%, 80%, or 90% sequence identity; or
j) SEQ ID NOs 83, 84, or 85 or an amino acid sequence having at least 60% sequence identity therewith, for example at least 70%, 80%, or 90% sequence identity; and/or
k) SEQ ID NOs 86, 87, or 88 or an amino acid sequence having at least 60% sequence identity therewith, for example at least 70%, 80%, or 90% sequence identity; and/or
l) SEQ ID NOs 89, 90, or 91 or an amino acid sequence having at least 60% sequence identity therewith, for example at least 70%, 80%, or 90% sequence identity.

6. An antibody or antigen-binding fragment thereof according to any of the preceding claims comprising a heavy chain variable region comprising the CDRs of SEQ ID NOS
a) SEQ ID NOs 65, 68, and 71;
b) SEQ ID NOs 66, 69, and 72;
c) SEQ ID NOs 67, 70, and 73;
d) SEQ ID NOs 11, 14, and 17;
e) SEQ ID NOs 12, 15, and 18;
f) SEQ ID NOs 13, 16, and 19;
g) SEQ ID NOs 29, 32, and 35;
h) SEQ ID NOs 30, 33, and 36;
i) SEQ ID NOs 31, 34, and 37;
j) SEQ ID NOs 47, 50, and 53;
k) SEQ ID NOs 48, 51, and 54;
l) SEQ ID NOs 49, 52, and 55;
m) SEQ ID NOs 83, 86, and 89;
n) SEQ ID NOs 84, 87, and 90; or
o) SEQ ID NOs 85, 88, and 91.

7. An antibody or antigen-binding fragment thereof according to any of the preceding claims comprising a heavy chain variable region having the amino acid sequence of SEQ ID NOs 7, 1, 3, 5, or 9, or an amino acid sequence having at least 60% sequence identity therewith, for example at least 70%, 80%, or 90% sequence identity.

8. An antibody or antigen-binding fragment thereof of any of the preceding claims comprising a light chain variable region comprising one or more of the CDRs of SEQ ID NOs 8, 2, 4, 6, or 10.

9. An antibody or antigen-binding fragment thereof of any of the preceding claims comprising a light chain variable region comprising the following CDRs:
a) SEQ ID NOs 74, 75, or 76 or an amino acid sequence having at least 60% sequence identity therewith, for example at least 70%, 80%, or 90% sequence identity; and/or
b) SEQ ID NOs 77, 78, or 79 or an amino acid sequence having at least 60% sequence identity therewith, for example at least 70%, 80%, or 90% sequence identity; and/or
c) SEQ ID NOs 80, 81, or 82 or an amino acid sequence having at least 60% sequence identity therewith, for example at least 70%, 80%, or 90% sequence identity; or
d) SEQ ID NOs 20, 21, or 22 or an amino acid sequence having at least 60% sequence identity therewith, for example at least 70%, 80%, or 90% sequence identity; and/or
e) SEQ ID NOs 23, 24, or 25 or an amino acid sequence having at least 60% sequence identity therewith, for example at least 70%, 80%, or 90% sequence identity; and/or
f) SEQ ID NOs 26, 27, or 28 or an amino acid sequence having at least 60% sequence identity therewith, for example at least 70%, 80%, or 90% sequence identity; or
g) SEQ ID NOs 38, 39, or 40 or an amino acid sequence having at least 60% sequence identity therewith, for example at least 70%, 80%, or 90% sequence identity; and/or
h) SEQ ID NOs 41, 42, or 43 or an amino acid sequence having at least 60% sequence identity therewith, for example at least 70%, 80%, or 90% sequence identity; and/or
i) SEQ ID NOs 44, 45, or 46 or an amino acid sequence having at least 60% sequence identity therewith, for example at least 70%, 80%, or 90% sequence identity; or
j) SEQ ID NOs 56, 57, or 58 or an amino acid sequence having at least 60% sequence identity therewith, for example at least 70%, 80%, or 90% sequence identity; and/or
k) SEQ ID NOs 59, 60, or 61 or an amino acid sequence having at least 60% sequence identity therewith, for example at least 70%, 80%, or 90% sequence identity; and/or
l) SEQ ID NOs 62, 63, or 64 or an amino acid sequence having at least 60% sequence identity therewith, for example at least 70%, 80%, or 90% sequence identity; or
m) SEQ ID NOs 92, 93, or 94 or an amino acid sequence having at least 60% sequence identity therewith, for example at least 70%, 80%, or 90% sequence identity; and/or
n) SEQ ID NOs 95, 96, or 97 or an amino acid sequence having at least 60% sequence identity therewith, for example at least 70%, 80%, or 90% sequence identity; and/or
o) SEQ ID NOs 98, 99, or 100 or an amino acid sequence having at least 60% sequence identity therewith, for example at least 70%, 80%, or 90% sequence identity.

10. An antibody or antigen-binding fragment thereof according to any of the preceding claims comprising a light chain variable region comprising the CDRs of SEQ ID NOS:
a) SEQ ID NOs 74, 77, and 80;
b) SEQ ID NOs 75, 78, and 81;
c) SEQ ID NOs 76, 79, and 82;
d) SEQ ID NOs 20, 23, and 26;
e) SEQ ID NOs 21, 24, and 27;
f) SEQ ID NOs 22, 25, and 28;
g) SEQ ID NOs 38, 41, and 44;
h) SEQ ID NOs 39, 42, and 45;
i) SEQ ID NOs 40, 43, and 46;
j) SEQ ID NOs 56, 59, and 62;
k) SEQ ID NOs 57, 60, and 63;
l) SEQ ID NOs 58, 61, and 64;
m) SEQ ID NOs 92, 95, and 98;
n) SEQ ID NOs 93, 96, and 99; or
o) SEQ ID NOs 94, 97, and 100.

11. An antibody or antigen-binding fragment thereof according to any of the preceding claims comprising a light chain variable region having the amino acid sequence of SEQ ID NOs 8, 2, 4, 6, or 10, or an amino acid sequence having at least 60% sequence identity therewith, for example at least 70%, 80%, or 90% sequence identity.

12. An antibody or antigen-binding fragment thereof according to any of the preceding claims comprising the following CDRs:
a) SEQ ID NOs 65, 68, 71, 74, 77, and 80 or SEQ ID NOs 66, 69, 72, 75, 78, and 81 or SEQ ID NOs 67, 70, 73, 76, 79, and 82 or an amino acid sequence having at least 60% sequence identity therewith, for example at least 70%, 80%, or 90% sequence identity; or
b) SEQ ID NOs 11, 14, 17, 20, 23, and 26 or SEQ ID NOs 12, 15, 18, 21, 24, and 27 or SEQ ID NOs 13, 16, 19, 22, 25, and 28 or an amino acid sequence having at least 60% sequence identity therewith, for example at least 70%, 80%, or 90% sequence identity;
c) SEQ ID NOs 29, 32, 35, 38, 41, and 44 or SEQ ID NOs 30, 33, 36, 39, 42, and 45 or SEQ ID Nos. 31, 34, 37, 40, 43, and 46 or an amino acid sequence having at least 60% sequence identity therewith, for example at least 70%, 80%, or 90% sequence identity;
d) SEQ ID NOs 47, 50, 53, 56, 59, and 62 or SEQ ID NOs 48, 51, 54, 57, 60, and 63 or SEQ ID NOs 49, 52, 55, 58, 61, and 64 or an amino acid sequence having at least 60% sequence identity therewith, for example at least 70%, 80%, or 90% sequence identity;
e) SEQ ID NOs 83, 86, 89, 92, 95, and 98 or SEQ ID NOs 84, 87, 90, 93, 96, and 99 or SEQ ID NOs 85, 88, 91, 94, 97, and 100 or an amino acid sequence having at least 60% sequence identity therewith, for example at least 70%, 80%, or 90% sequence identity.

13. An antibody or antigen-binding fragment thereof according to any of the preceding claims comprising:
a) a heavy chain variable region having the amino acid sequence of SEQ ID NO: 7 and a light chain variable region having the amino acid sequence of SEQ ID NO: 8, or an amino acid sequence having at least 60% sequence identity therewith, for example at least 70%, 80%, or 90% sequence identity; or
b) a heavy chain variable region having the amino acid sequence of SEQ ID NO: 1 and a light chain variable region having the amino acid sequence of SEQ ID NO: 2, or an amino acid sequence having at least 60% sequence identity therewith, for example at least 70%, 80%, or 90% sequence identity;
c) a heavy chain variable region having the amino acid sequence of SEQ ID NO: 3 and a light chain variable region having the amino acid sequence of SEQ ID NO: 4, or an amino acid sequence having at least 60% sequence identity therewith, for example at least 70%, 80%, or 90% sequence identity;
d) a heavy chain variable region having the amino acid sequence of SEQ ID NO: 5 and a light chain variable region having the amino acid sequence of SEQ ID NO: 6, or an amino acid sequence having at least 60% sequence identity therewith, for example at least 70%, 80%, or 90% sequence identity;
e) a heavy chain variable region having the amino acid sequence of SEQ ID NO: 9 and a light chain variable region having the amino acid sequence of SEQ ID NO: 10, or an amino acid sequence having at least 60% sequence identity therewith, for example at least 70%, 80%, or 90% sequence identity.

14. An antibody or antigen-binding fragment thereof according to any of the preceding claims wherein the antigen-binding fragment is selected from: scFv, Fv, Fab, F(ab′)2, Fab-SH, dsFv, sdAb, di-scFvs, and Fcabs.

15. An antibody or antigen-binding fragment thereof according to any of the preceding claims comprising a heavy chain constant region, or part thereof.

16. An antibody or antigen-binding fragment thereof wherein the heavy chain constant region is of an immunoglobulin subtype selected from the group consisting of IgG1, IgG2, IgG3 and IgG4.

17. An antibody or antigen-binding fragment thereof according to any of the preceding claims comprising a light chain constant region, or part thereof.

18. An antibody or antigen-binding fragment thereof according to claim 17 wherein the light chain constant region is of a kappa or lambda light chain.

19. An antibody or antigen-binding fragment thereof according to any of the preceding claims further comprising a moiety for increasing the in vivo half-life of the agent or a cytotoxic moiety.

20. An antibody or antigen-binding fragment thereof according to any of the preceding claims wherein the antibody or antigen-binding fragment is capable of inducing tumour specific immunity.

21. An isolated nucleic acid molecule encoding an antibody or antigen-binding fragment thereof according to any one of the preceding claims or a component polypeptide chain thereof.

22. A vector comprising a nucleic acid molecule according to claim 21, optionally wherein the vector is an expression vector.

23. A host cell comprising a nucleic acid molecule according to claim 21 or a vector according to claim 22, optionally wherein the host cell is a bacterial cell, a mammalian cell, or a human cell.

24. A pharmaceutical composition comprising an effective amount of an antibody or antigen-binding fragment thereof as defined in any one of the preceding claims and a pharmaceutically-acceptable buffer, diluent, carrier or excipient, optionally adapted for parenteral delivery or intravenous delivery.

25. A kit comprising an antibody or antigen-binding fragment thereof according to any one of the preceding claims or a pharmaceutical composition as defined in claim 24.

26. An antibody or antigen-binding fragment thereof according to any of the preceding claims for use in medicine.

27. An antibody or antigen-binding fragment thereof for use according to claim 26, wherein the antibody or antigen-binding fragment is for use in treating cancer in an individual.

28. An antibody or antigen-binding fragment thereof for use according to claim 27, wherein the cancer is a solid tumour, optionally wherein the solid tumour is selected from: sarcoma, lymphoma, blastoma, melanoma, carcinoma, lung cancer, pancreatic cancer, breast cancer, gastrointestinal cancer, head and neck cancer, brain cancer, liver cancer, ovarian cancer, prostate cancer, and cervical cancer.

29. An antibody or antigen-binding fragment thereof for use according to claim 27, wherein the cancer is a myeloid malignancy and/or neoplasm or a lymphoid malignancy and/or neoplasm, optionally selected from myelodysplastic syndrome and/or myelodysplastic neoplasms (MDS), myeloproliferative neoplasms (MPN), mastocytosis, myelodysplastic/myeloproliferative neoplasms, myeloma, and lymphoma, optionally wherein the myeloid malignancy and/or neoplasm is Acute Myeloid Leukemia (AML).

30. Use of an antibody or antigen-binding fragment thereof as defined in any one of claims 1 to 20:
(a) in the preparation of a medicament for inducing cell death and/or inhibiting the growth and/or proliferation of tumour cells;
(b) in the preparation of a diagnostic agent for detecting cells associated with a tumour, wherein the cells express SLAMF6; or
(c) for detecting cells associated with a tumour, wherein the cells express SLAMF6.

31. A method for inducing cell death and/or inhibiting the growth and/or proliferation of cells associated with a tumour in an individual in order to treat or prevent said tumour, comprising the step of administering to the individual an effective amount of an agent as defined in any one of claims 1-20, or a pharmaceutical composition as defined in claim 24.

32. A method for detecting cells associated with a tumour in an individual, comprising the step of administering to the individual an effective amount of an agent as defined in any one of claims 1 to 20, or a pharmaceutical composition as defined in claim 24 wherein the cells express SLAMF6.

33. A use according to claim 30, or the methods of claims 31-32 wherein the tumour is a cancer, optionally wherein the tumour is a solid tumour or a liquid tumour.

34. A use according to claim 33, wherein the solid tumour is selected from: sarcoma, lymphoma, blastoma, melanoma, carcinoma, lung cancer, pancreatic cancer, breast cancer, gastrointestinal cancer, colorectal cancer, head and neck cancer, brain cancer, liver cancer, ovarian cancer, prostate cancer, and cervical cancer; or wherein the liquid tumour is a myeloid malignancy/neoplasm or a lymphoid malignancy/neoplasm, optionally selected from myelodysplastic syndrome and/or myelodysplastic neoplasms (MDS), myeloproliferative neoplasms (MPN), mastocytosis, and myelodysplastic/myeloproliferative neoplasms, myeloma, and lymphoma, optionally wherein the myeloid malignancy and/or neoplasm is Acute Myeloid Leukemia (AML).
